SEQUENCE LISTING (17 sequences)

SEQ ID NO: 1 - protein, 267 aa, Thermotoga neapolitana
  MISC_FEATURE (258)..(267): HA tag
MAEVVLTDIGATDITFKGFPVTMELNFWNVKSYEGETWLKFDGEKVQFYADIYNIVLQNPDSWVHGYPEIYYGYKPWAAHNSGTEILPVKVKDLPDFYVTLDYSIWYENDLPINLAMETWITRKPDQTSVSSGDVEIMVWFYNNILMPGGQKVDEFTTTIEINGSPVETKWDVYFAPWGWDYLAFRLTTPMKDGRVKFNVKDFVEKAAEVIKKHSTRVENFDEMYFCVWEIGTEFGDPNTTAAKFGWTFKDFSVEIGEYPYDVPDYA

SEQ ID NO: 2 - DNA, 812 bp, Artificial sequence
  Codon usage optimized sequence encoding CelB di Thermotoga neapolitana with HA tag
  misc_feature (777)..(803): HA tag
ccatggctgaagttgttttaacagatattggtgctacagatattacatttaaaggttttccagttacaatggaattaaatttttggaatgttaaatcatatgaaggtgaaacatggttaaaatttgatggtgaaaaagttcaattttatgctgatatttataatattgttttacaaaatccagattcatgggttcatggttatccagaaatttattatggttataaaccttgggctgctcataattcaggtacagaaattttaccagttaaagttaaagatttaccagatttttatgttacattagattattcaatttggtatgaaaatgatttaccaattaatttagctatggaaacatggattacacgtaaaccagatcaaacatcagtttcatcaggtgatgttgaaattatggtttggttttataataatattttaatgccaggtggtcaaaaagttgatgaatttacaacaacaattgaaattaatggttcaccagttgaaacaaaatgggatgtttattttgctccttggggttgggattatttagcttttcgtttaacaacaccaatgaaagatggtcgtgttaaatttaatgttaaagattttgttgaaaaagctgctgaagttattaaaaaacattcaacacgtgttgaaaattttgatgaaatgtatttttgtgtttgggaaattggtacagaatttggtgatccaaatacaacagctgctaaatttggttggacatttaaagatttttcagttgaaattggtgaatatccatatgatgttccagattatgcttgagcatgc

SEQ ID NO: 3 - protein, 676 aa, Caldicellulosiruptor saccharolyticus
  MISC_FEATURE (668)..(776): HA tag
MGVTTSSPTPTPTPTVTVTPTPTPTPTPTVTATPTPTPTPVSTPATGGQIKVLYANKETNSTTNTIRPWLKVVNSGSSSIDLSRVTIRYWYTVDGERAQSAVSDWAQIGASNVTFKFVKLSSSVSGADYYLEIGFKSGAGQLQPGKDTGEIQIRFNKSDWSNYNQGNDWSWLQSMTSYGENEKVTAYIDGVLVWGQEPSGATPAPTMTVAPTATPTPTLSPTVTPTPAPTQTAIPTPTLTPNPTPTSSIPDDTNDDWLYVSGNKIVDKDGRPVWLTGINWFGYNTGTNVFDGVWSCNLKDTLAEIANRGFNLLRVPISAELILNWSQGIYPKPNINYYVNPELEGKNSLEVFDIVVQTCKEVGLKIMLDIHSIKTDAMGHIYPVWYDEKFTPEDFYKACEWITNRYKNDDTIIAFDLKNEPHGKPWQDTTFAKWDNSTDINNWKYAAETCAKRILNINPNLLIVIEGIEAYPKDDVTWTSKSSSDYYSTWWGGNLRGVRKYPINLGKYQNKVVYSPHDYGPSVYQQPWFYPGFTKESLLQDCWRPNWAYIMEENIAPLLIGEWGGHLDGADNEKWMKYLRDYIIENHIHHTFWCFNANSGDTGGLVGYDFTTWDEKKYSFLKPALWQDSQGRFVGLDHKRPLGTNGKNINITTYYNNNEPEPVPASKYPYDVPDYA

SEQ ID NO: 4 - DNA, 2039 bp, Artificial sequence
  Codon-optimized sequence of CBM3GH5 from Caldicellulosiruptor saccharolyticus with HA tag
  misc_feature (2004)..(2030): HA tag
ccatgggtgttacaacatcatcaccaacaccaacaccaacaccaacagttacagttacaccaacaccaacaccaacaccaacaccaacagttacagctacaccaacaccaacaccaacaccagtttcaacaccagctacaggtggtcaaattaaagttttatatgctaataaagaaacaaattcaacaacaaatacaattcgtccttggttaaaagttgttaattcaggttcatcatcaattgatttatcacgtgttacaattcgttattggtatacagttgatggtgaacgtgctcaatcagctgtttcagattgggctcaaattggtgcttcaaatgttacatttaaatttgttaaattatcatcatcagtttcaggtgctgattattatttagaaattggttttaaatcaggtgctggtcaattacaaccaggtaaagatacaggtgaaattcaaattcgttttaataaatcagattggtcaaattataatcaaggtaatgattggtcatggttacaatcaatgacatcatatggtgaaaatgaaaaagttacagcttatattgatggtgttttagtttggggtcaagaaccatcaggtgctacaccagctccaacaatgacagttgctccaacagctacaccaacaccaacattatcaccaacagttacaccaacaccagctccaacacaaacagctattccaacaccaacattaacaccaaatccaacaccaacatcatcaattccagatgatacaaatgatgattggttatatgtttcaggtaataaaattgttgataaagatggtcgtccagtttggttaacaggtattaattggtttggttataatacaggtacaaatgtttttgatggtgtttggtcatgtaatttaaaagatacattagctgaaattgctaatcgtggttttaatttattacgtgttccaatttcagctgaattaattttaaattggtcacaaggtatttatccaaaaccaaatattaattattatgttaatccagaattagaaggtaaaaattcattagaagtttttgatattgttgttcaaacatgtaaagaagttggtttaaaaattatgttagatattcattcaattaaaacagatgctatgggtcatatttatccagtttggtatgatgaaaaatttacaccagaagatttttataaagcttgtgaatggattacaaatcgttataaaaatgatgatacaattattgcttttgatttaaaaaatgaaccacatggtaaaccttggcaagatacaacatttgctaaatgggataattcaacagatattaataattggaaatatgctgctgaaacatgtgctaaacgtattttaaatattaatccaaatttattaattgttattgaaggtattgaagcttatccaaaagatgatgttacatggacatcaaaatcatcatcagattattattcaacatggtggggtggtaatttacgtggtgttcgtaaatatccaattaatttaggtaaatatcaaaataaagttgtttattcaccacatgattatggtccatcagtttatcaacaaccttggttttatccaggttttacaaaagaatcattattacaagattgttggcgtccaaattgggcttatattatggaagaaaatattgctccattattaattggtgaatggggtggtcatttagatggtgctgataatgaaaaatggatgaaatatttacgtgattatattattgaaaatcatattcatcatacattttggtgttttaatgctaattcaggtgatacaggtggtttagttggttatgattttacaacatgggatgaaaaaaaatattcatttttaaaaccagctttatggcaagattcacaaggtcgttttgttggtttagatcataaacgtccattaggtacaaatggtaaaaatattaatattacaacatattataataataatgaaccagaaccagttccagcttcaaaatatccatatgatgttccagattatgcttgagcatgc

SEQ ID NO: 5 - protein, 482 aa, Pyrococcus furiosus
  MISC_FEATURE (474)..(482): HA tag
MAKFPKNFMFGYSWSGFQFEMGLPGSEVESDWWVWVHDKENIASGLVSGDLPENGPAYWHLYKQDHDIAEKLGMDCIRGGIEWARIFPKPTFDVKVDVEKDEEGNIISVDVPESTIKELEKIANMEALEHYRKIYSDWKERGKTFILNLYHWPLPLWIHDPIAVRKLGPDRAPAGWLDEKTVVEFVKFAAFVAYHLDDLVDMWSTMNEPNVVYNQGYINLRSGFPPGYLSFEAAEKAKFNLIQAHIGAYDAIKEYSEKSVGVIYAFAWHDPLAEEYKDEVEEIRKKDYEFVTILHSKGKLDWIGVNYYSRLVYGAKDGHLVPLPGYGFMSERGGFAKSGRPASDFGWEMYPEGLENLLKYLNNAYELPMIITENGMADAADRYRPHYLVSHLKAVYNAMKEGADVRGYLHWSLTDNYEWAQGFRMRFGLVYVDFETKKRYLRPSALVFREIATQKEIPEELAHLADLKFVTRKYPYDVPDYA

SEQ ID NO: 6 - DNA, 1457 bp, Artificial sequence
  Codon optimized seqeunce encoding for beta glucosidase from Pyrococcus furiosus with HA tag
  misc_feature (1443)..(1457): HA tag
ccatggctaaatttccaaaaaattttatgtttggttattcatggtcaggttttcaatttgaaatgggtttaccaggttcagaagttgaatcagattggtgggtttgggttcatgataaagaaaatattgcttcaggtttagtttcaggtgatttaccagaaaatggtccagcttattggcatttatataaacaagatcatgatattgctgaaaaattaggtatggattgtattcgtggtggtattgaatgggctcgtatttttccaaaaccaacatttgatgttaaagttgatgttgaaaaagatgaagaaggtaatattatttcagttgatgttccagaatcaacaattaaagaattagaaaaaattgctaatatggaagcattagaacattatcgtaaaatttattcagattggaaagaacgtggtaaaacatttattttaaatttatatcattggccattaccattatggattcatgatccaattgctgttcgtaaattaggtccagatcgtgctccagctggttggttagatgaaaaaacagttgttgaatttgttaaatttgctgcttttgttgcttatcatttagatgatttagttgatatgtggtcaacaatgaatgaaccaaatgttgtttataatcaaggttatattaatttacgttcaggttttccaccaggttatttatcatttgaagctgctgaaaaagctaaatttaatttaattcaagctcatattggtgcttatgatgctattaaagaatattcagaaaaatcagttggtgttatttatgcttttgcttggcatgatccattagctgaagaatataaagatgaagttgaagaaattcgtaaaaaagattatgaatttgttacaattttacattcaaaaggtaaattagattggattggtgttaattattattcacgtttagtttatggtgctaaagatggtcatttagttccattaccaggttatggttttatgtcagaacgtggtggttttgctaaatcaggtcgtccagcttcagattttggttgggaaatgtatccagaaggtttagaaaatttattaaaatatttaaataatgcttatgaattaccaatgattattacagaaaatggtatggctgatgctgctgatcgttatcgtccacattatttagtttcacatttaaaagctgtttataatgctatgaaagaaggtgctgatgttcgtggttatttacattggtcattaacagataattatgaatgggctcaaggttttcgtatgcgttttggtttagtttatgttgattttgaaacaaaaaaacgttatttacgtccatcagctttagtttttcgtgaaattgctacacaaaaagaaattccagaagaattagctcatttagctgatttaaaatttgttacacgtaaatatccatatgatgttccagattatgcttaagcatgc

SEQ ID NO: 7 - protein, 1036 aa, Thermotoga neapolitana
  MISC_FEATURE (1028)..(1036): HA tag
MATGALGFGGKGVSPFETVLVLSFEGNTDGASPFGKDVVVTASQDVAADGEYSLKVENRTSVWDGVEIDLTGKVNTGTDYLLSFHVYQTSDSPQLFSVLARTEDEKGERYKILADKVVVPNYWKEILVPFSPTFEGTPAKFSLIITSPKKTDFVFYVDNVQVLTPKEAGPKVVYETSFEKGIGDWQPRGSDVKISISPKVAHSGKKSLFVSNRQKGWHGAQISLKGILKTGKTYAFEAWVYQESGQDQTIIMTMQRKYSSDSSTKYEWIKAATVPSGQWVQLSGTYTIPAGVTVEDLTLYFESQNPTLEFYVDDVKVVDTTSAEIKLEMNPEEEIPALKDVLKDYFRVGVALPSKVFINQKDIALISKHFNSITAENEMKPDSLLAGIENGKLKFRFETADKYIEFAQQNGMVVRGHTLVWHNQTPEWFFKDENGNLLSKEEMTERLREYIHTVVGHFKGKVYAWDVVNEAVDPNQPDGLRRSTWYQIMGPDYIELAFKFAREADPNAKLFYNDYNTFEPKKRDIIYNLVKSLKEKGLIDGIGMQCHISLATDIRQIEEAIKKFSTIPGIEIHITELDISVYRDSTSNYSEAPRTALIEQAHKMAQLFKIFKKYSNVITNVTFWGLKDDYSWRATRRNDWPLIFDKDYQAKLAYWAIVAPEVLPPLPKESKISEGEAVVVGMMDDSYMMSKPIEIYDEEGNVKATIRAIWKDSTIYVYGEVQDATKKPAEDGVAIFINPNNERTPYLQPDDTYVVLWTNWKSEVNREDVEVKKFVGPGFRRYSFEMSITIPGVEFKKDSYIGFDVAVIDDGKWYSWSDTTNSQKTNTMNYGTLKLEGVMVATAKYGTPVIDGEIDDIWNTTEEIETKSVAMGSLEKNATAKVRVLWDEENLYVLAIVKDPVLNKDNSNPWEQDSVEIFIDENNHKTGYYEDDDAQFRVNYMNEQSFGTGASAARFKTAVKLIEGGYIVEAAIKWKTIKPSPNTVIGFNVQVNDANEKGQRVGIISWSDPTNNSWRDPSKFGNLRLIKYPYDVPDYA

SEQ ID NO: 8 - DNA, 3119 bp, Artificial sequence
  Codon optimized sequence encoding xylanase XynA from Thermotoga neapolitana with HA tag
ccatggctacaggtgctttaggttttggtggtaaaggtgtttcaccatttgaaacagttttagttttatcatttgaaggtaatacagatggtgcttcaccatttggtaaagatgttgttgttacagcttcacaagatgttgctgctgatggtgaatattcattaaaagttgaaaatcgtacatcagtttgggatggtgttgaaattgatttaacaggtaaagttaatacaggtacagattatttattatcatttcatgtttatcaaacatcagattcaccacaattattttcagttttagctcgtacagaagatgaaaaaggtgaacgttataaaattttagctgataaagttgttgttccaaattattggaaagaaattttagttccattttcaccaacatttgaaggtacaccagctaaattttcattaattattacatcaccaaaaaaaacagattttgttttttatgttgataatgttcaagttttaacaccaaaagaagctggtccaaaagttgtttatgaaacatcatttgaaaaaggtattggtgattggcaaccacgtggttcagatgttaaaatttcaatttcaccaaaagttgctcattcaggtaaaaaatcattatttgtttcaaatcgtcaaaaaggttggcatggtgctcaaatttcattaaaaggtattttaaaaacaggtaaaacatatgcttttgaagcttgggtttatcaagaatcaggtcaagatcaaacaattattatgacaatgcaacgtaaatattcatcagattcatcaacaaaatatgaatggattaaagctgctacagttccatcaggtcaatgggttcaattatcaggtacatatacaattccagctggtgttacagttgaagatttaacattatattttgaatcacaaaatccaacattagaattttatgttgatgatgttaaagttgttgatacaacatcagctgaaattaaattagaaatgaatccagaagaagaaattccagctttaaaagatgttttaaaagattattttcgtgttggtgttgctttaccatcaaaagtttttattaatcaaaaagatattgctttaatttcaaaacattttaattcaattacagctgaaaatgaaatgaaaccagattcattattagctggtattgaaaatggtaaattaaaatttcgttttgaaacagctgataaatatattgaatttgctcaacaaaatggtatggttgttcgtggtcatacattagtttggcataatcaaacaccagaatggttttttaaagatgaaaatggtaatttattatcaaaagaagaaatgacagaacgtttacgtgaatatattcatacagttgttggtcattttaaaggtaaagtttatgcttgggatgttgttaatgaagctgttgatccaaatcaaccagatggtttacgtcgttcaacatggtatcaaattatgggtccagattatattgaattagcttttaaatttgctcgtgaagctgatccaaatgctaaattattttataatgattataatacatttgaaccaaaaaaacgtgatattatttataatttagttaaatcattaaaagaaaaaggtttaattgatggtattggtatgcaatgtcatatttcattagctacagatattcgtcaaattgaagaagctattaaaaaattttcaacaattccaggtattgaaattcatattacagaattagatatttcagtttatcgtgattcaacatcaaattattcagaagctccacgtacagctttaattgaacaagctcataaaatggctcaattatttaaaatttttaaaaaatattcaaatgttattacaaatgttacattttggggtttaaaagatgattattcatggcgtgctacacgtcgtaatgattggccattaatttttgataaagattatcaagctaaattagcttattgggctattgttgctccagaagttttaccaccattaccaaaagaatcaaaaatttcagaaggtgaagctgttgttgttggtatgatggatgattcatatatgatgtcaaaaccaattgaaatttatgatgaagaaggtaatgttaaagctacaattcgtgctatttggaaagattcaacaatttatgtttatggtgaagttcaagatgctacaaaaaaaccagctgaagatggtgttgctatttttattaatccaaataatgaacgtacaccatatttacaaccagatgatacatatgttgttttatggacaaattggaaatcagaagttaatcgtgaagatgttgaagttaaaaaatttgttggtccaggttttcgtcgttattcatttgaaatgtcaattacaattccaggtgttgaatttaaaaaagattcatatattggttttgatgttgctgttattgatgatggtaaatggtattcatggtcagatacaacaaattcacaaaaaacaaatacaatgaattatggtacattaaaattagaaggtgttatggttgctacagctaaatatggtacaccagttattgatggtgaaattgatgatatttggaatacaacagaagaaattgaaacaaaatcagttgctatgggttcattagaaaaaaatgctacagctaaagttcgtgttttatgggatgaagaaaatttatatgttttagctattgttaaagatccagttttaaataaagataattcaaatccttgggaacaagattcagttgaaatttttattgatgaaaataatcataaaacaggttattatgaagatgatgatgctcaatttcgtgttaattatatgaatgaacaatcatttggtacaggtgcttcagctgctcgttttaaaacagctgttaaattaattgaaggtggttatattgttgaagctgctattaaatggaaaacaattaaaccatcaccaaatacagttattggttttaatgttcaagttaatgatgctaatgaaaaaggtcaacgtgttggtattatttcatggtcagatccaacaaataattcatggcgtgatccatcaaaatttggtaatttacgtttaattaaatatccatatgatgttccagattatgcttaagcatgc

SEQ ID NO: 9 - DNA, 8270 bp, Artificial sequence
  Expression vector pCLE
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgctgcagccaacaagttttaaaacctcttcaacgctatattccacaaaacggtggatttacatggccaggtgattatttacgtttagaaattgttgaaatgccaaaattaaaatcaatcaatattaaaaaaacaagtttaaaacaaaaaattaatgttcaaccagttggtattatgccacgtaaatatttaattgaaaaacataatattaaagtattgaaaaagaaactttctcaagcttattcaacacaacaattaactaaagtggtacaagaatataaaaatttaattcaaaattctccaccagctatttaaaaatatgccatatttttactaattttatattccaagacattgtatgcttataaataaagaagatgctttgcatctctaaagatgagtacaatgttttggaatatttatataatatattaatatatatagttaaatgaaaaaactaaaaaataagcgttagtgaataatactttttatatataaattctactatatgtttgagcttccttttatataaattttaaatttataacaagttacactctcagtatttcctgcctttttgaaagtaaagccacaatgtttacatactaggcagtggcgataccactgccactggcgtcctccttcggagtatgtaaacatgctaagtttacttgcccgaaggggaaggaggaggttcttatttcaaattacctaagataaagtttccttcggggtttatacgtaagaatttaagtaaaataagttaaaatatgaatgccttggggcatattaattccacttactatgagtgattctattgaaacaaaaaacatgggacgtattgtacaaattattggtcctgttttagatatcgtatttgctaaaggccaagtaccaaatatttacaacgctctaactattcgtgctaaaaactcagcaggtacagaaatggctgttacttgtgaagttcaacaacttttaggtgataactgtgtacgtgcagtttctatgaacccaacagaaggtttaatgcgtggtatggaagttgtagatactggtaaaccgttaagtgttcctgtaggtaaagtaactttaggacgtattttcaacgttctaggtgaacctgtagataacatgggtaatgttaaagttgaagaaactttaccaattcaccgtacagctcctgctttcgttgatttagatactcgtttatcaatttttgaaacaggtattaaagttgtagaccttttagctccataccgtcgtggtggtaaaattggtcttttcggtggtgctggtgtaggcaaaacagttttaattatggaactgatcaacaacattgctaaagcacacggtggtgtttctgtatttgctggtgttggtgaacgtacacgtgaaggtaacgacctttacacagaaatgaaagaatctggtgttattgttgaaaagaacttatctgattcaaaagtagctcttgtatacggtcaaatgaacgaaccaccaggtgctcgtatgcgtgttgctttaacagctttaacaatggctgaatatttccgtgatgttaacaaacaagacgtattattcttcattgataacattttccgtttcgtacaagctggtgctgaagtatctgctttattaggtcgtatgccatcagctgtaggttaccaacctactttagctacagaaatgggtggtcttcaagaacgtattacttcaactaaagatggttctatcacatcgattcaggctgtatatgtacctgcagatgaccttactgaccctgctcctgctacaacattcgctcacttagatgctactacagtactttctcgtaacttagctgctaaaggtatttaccctgcggttgacccattagaatcaacttcaactatgttacaaccgtggattcttggtgaaaaacactatgattctgcacaaagcgtaaaaaaaactttacaacgttacaaagaattacaagatattattgctattctaggtttagatgaattatctgaagaagacagacttattgtagctcgtgctcgtaaaattgaacgtttcttatcacaaccattcttcgtagctgaagtatttacaggttcacctggtaaatatgtttctttagctgaaactattgaaggttttggtaaaattttcgctggtgaattagatgatttaccagaacaagcattctacttagtaggtaacattacagaagctattagtaaagctgcttcattaaaatttttatttttcatgatgtttatgtgaataatttggaaccttcacgcagatgctcatgactttgacagtcatacaagtgatctagaagaaatttctagaaaagtattcagtgcacactttggtcaattaggtatcattttcatttggttaagtgggtgcgacacgaagacgtatatatttttatagtttaaaaagatacttttacactgtagttgaaaagtataagcacttttaaaaatcaaagcagtataaggcaattatttgcattttgctttagttctttttattttttcagaaaaacttactttttagtttcaattatgtctaaaaggatcccgggtaccgagctccaccgcggtggcggccgctctagctagaactagtggatcgcactctaccgattgagttacatccgctttagtatgttactatttcttttattataacttataaaatataatacataaagataaattctataataaaaagctaagattttatttttctggcacatcgtaatttataaagacaggcaaatttaaacaaaagataactttagaacttaattttaaaaatgtaaaatgatgtttaggtatttaacctaaacaccataaaaaataaaaacgatgtttatgctattcacataaacatcatgaaaaataaaaattaaagtttgtcaatagtatcaaattcgaatttaatttctttccaaacttcacatgcagcagcaagttctggagaccatttacaagctgaacgaattacgtcgccaccttcacgagcaaggtcacgaccttcgttacgagcttgagtacaagcttgcatgcctgcagttatttgccaactaccttagtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgataaccgcttcacgagccatggatttctccttataataacaattatttaatttaataaatacattatttacaccattagcaacgcttttaattattatgtgttcataaaaaatgcatggttattaaattagacaaatagttttttttacatcatatgttatattataccataaatatattttttattggtttacaaattatttttacttattttaaagatattgttttattttttaccatatgtgtattcgtaaaagtataacattaaacagtatttacttaaaaatgttgaattaagaaagcttatcgataagcttgatctagagtcgataatacgaatagtaatacggttttataataataataataataataataataataataataataataataaaattcttaaaaatatacaaaaaattaacatacaaatttaatcccctccgggattttaaatactccgtaaggaggacgtcctaactacggattgaaatccctaagtttactctttctcggggaaggggaaggggacgtcctaaaatacatttcttcaactgccctttctttcctcaccccttcggggacggctcataaagacaagctttcctctggtcccctttggggaaagagacatttcatttaaaagacaagctttggttgtcccctttggggaagcaactattttcaggcgaccgacgtatcctctagctcaaatttcttcccctttggggacgacgctcaatcatacccttccatcccctacggggatatataccctttcagggtattagggtatgggggaagccgatggactatttacattccttgtcccctttgggaatcggctttgccgatagatttgttgggaagtagggtagggatgtaaagcttgtcaaagtttactaaaaaaaagtaatatataatatatttaaaaaaagtaatatataatatatttaaaaaaagtaatatataatatatttaaaaaaagtaatatataatatatttaaaaaaagtaatatataatatatttaaaaaaagtaatatataatatatttaaatgtatttaaaaatttttaagatttttaaattatatttccggacagattattttaggatcgacaacaaaagttacatttatttatatttatgacaaataatgatatttatagcgtacaagacagtttagtagaacattttaatggtttgaatataaaaacagaaaaccagtttaaacttttagtaaataagtactgcttgtatctaaaaaattcgacagaaggtcttatttttttgtttaagctatatgtaaacagttctactcgtgagcaatctattattcaccttagtatcttgaattggttaaatactaaatctttaccccaaagaaaagttactctaattacctttttacaagatttagatttagaatttggtactgtaaattatctacagcaaactgaagtattggttgttttaaaatttatctatagtttactaattaagaaaaaccaagatcgagcatcgcgagatactccatcagaggatcctctagaggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 10 - DNA, 13840 bp, Artificial sequence
  Expression vector pLM20
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcaaacgaagaaaaacgaagttttaacagttaaaactgcgttaaatatgtttttaagaaattatacgagctaaccttcactaagcttcaggaacatttggggagttttgctgctttgcagccccgaaggggacgacatttgatgagtttcaatgacgttcgatgacgttcgatgacactttacaatatgtcacgaaggttgaccaaaaggtatgaccgaaggtaatagcaacattgttgtgttttaaggtctataaataaaggggacttgttttttttttacaactttgttataataaaataaatattattttaaagtttgaataatcaagtttgcttaataaaaatataaaatttttccccttacgggtgtaaataacattaaatccctttatgactacaaaaaaaacaatacaattttatgctaatatagctcaggttcgtaatcacgaactaatttctcttagtaacatccaaacaccttctcaaggttcaattacaattttttgtaaaacttgtaaaaccagctttacaacaacagcacgttcttatcagaacgctcgtaaaacaggttgtcctcaatgtaaagcaaaaacaacaagtgaaaactggaaaggtaaaattcgtacaaaatctcctgaagaggcctctaaacaggccgttttaaatgaatataaacaacaaaaacacttacaaaaaggtttagcatatgcacatatttctaataaagaagatttaaaaatttttctgaaagaaagtccaaacgtttataacaattttatactccaacggattgaccatccagttataggaaaatatactgaaaaccatcatattattcctaaacacaccggtggtccacataaacgctggaatttaataaaattaacaccagaagaccatatggaagctcaccgattacgtgctttagtatataacgaagctggtgatcaccaagcaattcgtttcagaactaatccttctgagctagttgaacgacgtcttcgaggaaaccaaatagccaacgaaacgcgtttaagagaacgtactggtatctatgccgaaggcgcttcttctaaaggtggacgtattggtggtctagtaaaaagtcacgaaaaagatttaaaacaaagtacaaaaatgagccaacccgtattaaaagctctttacgaaggttcacgttggaaacatttacaatccggaactgaactaaaccttcaaccaaacagattgtttacgcttccgcagctagttcaaaaacttttagaagctttaccaccatgtaaagataaagaagttttagaaagagcaaaaacaagtacagttacttcaaatctggctcgtgtaattaaaaaacaacgaccttctgcatatggctggataacgttttaaacctttttacgttttttgtaataatatggcaacctgcagcgaagctagggatgggcattgcttccctagaacgttcagagactagagcttgagtcccaacaataatagctcactagcgcccggcatctaaaatggtttaaattttagatggtgatatagtccaacccaccttgaaaaaggtggacgacgaccagtagattaaaaaggtttttagtctatattaaatggttactttggtcgtctaatcttccaatacgcttctttcaacaactctcgttcattacacttcttcttagctgcttggccggtaatcggtatttggttcactgctttaggtttatcaactatggcattcaacttaaacggtttcaacttcaaccaatcagtagtagactcacaaggtcgtgtactaaacacttgggcagacatcatcaaccgtgctaacttaggtatggaagtaatgcacgagcgtaacgctcacaacttccctctagacttagcttcaactaactctagctcaaacaactaatttttttttaaactaaaataaatctggttaaccatacctggtttattttagtttatacacacttttcatatatatatacttaatagctaccataggcagttggcaggacgtccccttacgggacaaatgtatttattgttgcctgccaactgcctaatataaatattagtggacgtccccttccccttacgggcaagtaaacttagggattttaatgctccgttaggaggcaaataaattttagtggcagttgcctcgcctatcggctaacaagttccttcggagtatataaatatcctgccaactgccgatatttatatactaggcagtggcggtaccactcgactaatatttatattccgtaagacgtcctccttcggagtatgtaaacatgctaagtttacttgcccaatatttatattaggacgtcagtggcagtggaccaccactcgtattttatactcgaaaggcagttggcaggcaactcgactaaaatttatttacccgaagacgtcccgaagaaggggaaggaggcagtggtaccaccactggctccgcagtattaacatcctatatttatatactccgaagaacttcttagccgatggcaactgccacaaaaaaccatcggcttcgtttcacacagatggtgagaaccacacgtttcgtcctataaaaatagctaagtttacttgcccaatatttatattaggacgtccccttcggtaaataaattttagtggctgtgtgacgttcactggcgtcttggtaggttctgtgactgactaaataaaaaagtatttgtcgtctacgatatgtaaatctgtcgtatacgatatgtaaatttgagctcttatggcctctacatcgaggtttattatcttaccgaaggtaaatgccttcgtggatcttatgggacgtcctgtgtccttcctagtggtcaataatcacttcgtgacagcctgggcttacatttatataagcgctgttatatttatacgctgttagacaaggtttaaatacataaatttttattagtctatcgaccgttaattgcttaacttaccgaaggttaatcgctttacttaccgaaggttaatcgctttacttaccgaaggttaattgcttaacttactgaaggttaatcgctttacttaccgaaggttaattgcttaacttactgaaggttaatcgctttacttaccgaaggttaatccaatttttttttcgccatatgtagacgtttaattgctacaacgttattagcctttcgtcgctatcaaatcggttcagatatatatcactttattcactttcgtttatattatggctggattaggtcttttagttaattaaaatttacatatttaatgctatttattattattgcaattgcattaaatatttttttaaaaaaaattaatcttcagctatattagtaaataacccataaatagtttcaattggaataattggaattggatatggactagttttattttcttctaataactttaatatcgctggatcttaatacgactcactatagggcgaattggagctccaccgcggtggcggcccgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgggccgctctagctagaactagtggatcgcactctaccgattgagttacatccgctttagtatgttactatttcttttattacttataaaatataatacataaagataaattctataataaaaagctaagattttatttttctggcacatcgtaatttataaagcacaggcaaatttaaacaaaagataactttagaacttaattttaaaaatgtaaaatgatgtttaggtatttaacctaaacaccataaaaaataaaaacgatgtttatgctattcacataaacatcatgaaaaataaaaattaaagtttgtcaatagtatcaaattcgaatttaatttctttccaaacttcacatgcagcagcaagttctggagaccatttacaagctgaacgaattacgtcgccaccttcacgagcaaggtcacgaccttcgttacgagcttgagtacaagcttgcatgcctgcagttatttgccaactaccttagtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgataaccgcttcacgagccatggacattttcacttctggagtgtattgttcaattaaatctttaataagattactaagttcttctggagtacgcattgccataaaaaagaaaaaataaataaaagattaaaaaagtttatttttaaaatctttctcgagaattttaaataagtttaaaattcaacaaaaatagtgagtggtaagatcacttgttaacaaaagtaatggttcacccttgtcatatttaaatactaaaattcatttgcccgaagaggacaaatttatttattgcattaaaatccctaagtttacttgcccgtaaggggaagggggggacgtccacaggcgtcgtaagcaactaaagtttatgacgccgattgctttgttaggaaaatataaatatcccataagaaaaggtcctttaaaggttttatggactaaataaaaaagatagcataagcattaaaatcatgcaaattaaaaaaaaggtaaatgtatttataaaaaggtaaatgtatttatatagtatttatattatagcataataataaatatatttataaattgattgttcttagagctaaaagagaagaacaatgggtttataggtattttgagaccagttataaaaatgacttttgacgtttaggtatataaacactgcctctaataaagtcatcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgcgataccgtcgacctcgagggggggcccggtacccagcttttgttccctttagtgagggttaatcccgggtaccgagctccaccgcggtggcggccgctctagctagaactagtggatcgcactctaccgattgagttacatccgctttagtatgttactatttcttttattacttataaaatataatacataaagataaattctataataaaaagctaagattttatttttctggcacatcgtaatttataaagcacaggcaaatttaaacaaaagataactttagaacttaattttaaaaatgtaaaatgatgtttaggtatttaacctaaacaccataaaaaataaaaacgatgtttatgctattcacataaacatcatgaaaaataaaaattaaagtttgtcaatagtatcaaattcgaatttaatttctttccaaacttcacatgcagcagcaagttctggagaccatttacaagctgaacgaattacgtcgccaccttcacgagcaaggtcacgaccttcgttacgagcttgagtacaagcttgcatgcttataatgtgaaatcaactaatggagctgtaccacgtgtaccattttgaatatcaacaccttcagcatttgattttttaacagcaacttcaccttgatttgtttcaacttcgaataaaaaacgagcttgacgaccagctgtaatagaagcaccaacatcagcagctacatcagctggttttaatgtaacagcaccacctgatggtaatgtaactgataatgctttacgtgtaactaaaactgtaccttcaccacgaccattagcagcatgatcagcatattctgtaattgtaacgaaaattttaccaccttctgttggtaaaatgtttgaaacatcacgaatgtgaattaaatttgctgatgttgtacctgttgtaattgtatcgaatttcataccatcacgtgttaaaatgaataaatcagctaattcatctttgaaataattatatgttgcttgtgaatcaccttgaatttcagctttaacattgaagtttacagctggaactggaactgtattgtcagcagcaacatataatgtaccgaaagcagctaaagcattttgaacaccacctgccatattaccagcacctaaaacagctgtagctgattgaccatcagcagcagctttccaaccaattgaagcaccagctttattaactaaattaccattagcatcttttttaaaagctgaaacatcacctgttaaagttaaatttactttatcacgttgaattgtttgatttgttgtaacatttgtaatagcgaaatctgtagctgaaatattagcatcaccaatatttaattttaaaaaaccttctgaaactaaagctgtagctttaccagcatttgttaatgataattggaataatttacctaatttgaataatggttttgtagcaactggatcagctgtatatgatgcattccataaagcaccagctgttaaagcaactgtaccttgtgaacctgatgttaatttaaatgcttgattagctaagtttaattttaattcaacttcagcatcagcgaaattaattgtttcttcaacatctgaagctaaagctaaacgtaaacgtttaccatttgaatcaattgtgtgaacaattgttaattgtgtaccatctgaaataattttgaagaatttacgaacatcattaacttcacctgaattagcaaaattaccgtgatcatttggattaactaataaacctgtaaatggacgacgccaaaaaccatttgataaccattgacgaatttcagcatcattgaattgaccttcaccttgaatttctaaaacaatgaaaccattattttttaaatcaccaacttcagagaaatttaatgttaaattttgttgagctgttgaaaaatctaaaactggttgttttgtaactgaagctaatgatgttgtaacgaatgtattatcatttggtgaaataacaacatcagcaaaagctggagcagctgaaccaacaacaatagcagcagcaattaatgtttttttgaacatagccatggacattttcacttctggagtgtattgttcaattaaatctttaataagattactaagttcttctggagtacgcattgccataaaaaagaaaaaataaataaaagattaaaaaagtttatttttaaaatctttctcgagaattttaaataagtttaaaattcaacaaaaatagtgagtggtaagatcacttgttaacaaaagtaatggttcacccttgtcatatttaaatactaaaattcatttgcccgaagaggacaaatttatttattgcattaaaatccctaagtttacttgcccgtaaggggaagggggggacgtccacaggcgtcgtaagcaactaaagtttatgacgccgattgctttgttaggaaaatataaatatcccataagaaaaggtcctttaaaggttttatggactaaataaaaaagatagcataagcattaaaatcatgcaaattaaaaaaaaggtaaatgtatttataaaaaggtaaatgtatttatatagtatttatattatagcataataataaatatatttataaattgattgttcttagagctaaaagagaagaacaatgggtttataggtattttgagaccagttataaaaatgacttttgacgtttaggtatataaacactgcctctaataaagtcatcgatatcggatccgtatccatgctagcaatatctgatggtacttgcatttcataagtttggcctggaataaccaccgtttcggaagtacctgtcgctttaagttttatagctaaatctaaagtttctttaagtcttttagctgtattaaatactccacgactttcccttacgggacaataaataaatttgtccccttccccttacgtgacgtcagtggcagttgcctgccaactgcctccttcggagtattaaaatcctatatttatatactcctaagtttacttgcccaatatttatattaggcagttggcaggcaactgccactgacgtcccgaaggggaaggggaaggacgtccccttcgggtaaataaattttagtggcagtggtaccaccactgcctgcttcctccttccccttcgggcaagtaaacttagaataaaatttatttgctgcgctagcaggtttacatactcctaagtttacttgcccgaaggggaaggaggacgtccccttacgggaatataaatattagtggcagtggtacaataaataaattgtatgtaaaccccttcgggcaactaaagtttatcgcagtatataaatatagaatgtttacatactccgaaggaggacgccagtggcagtggtaccgccactgcctgtccgcagtattaacatcctattttaatactccgaaggaggcagttggcaggcaactgccactaatatttatattcccgtaaggggacgtcctaatttaatactccgaaggaggcagttggcaggcaactgccactaaaatttatttgcctcctaacggagcattaaaatcccgaaggggacgtcccgaaggggaaggggaaggaggcaactgcctgcttcctccttccccttcgggcaagtaaacttagaataaaatttatttgctgcgctagcaggtttacatactcctaagtttacttgcccgaaggggaaggaggacgtccccttacgggaatataaatattagtggcagtggtacaataaataaattgtatgtaaaccccttcgggcaactaaagtttatcgcagtatataaatatcggcagttggcaggcaactgccactaaaattcatttgcccgaaggggacgtccactaatatttatattcccgtaaggggacgtcccgaaggggaaggggacgtcctaaacggagcattaaaatccctaagtttacttgcctaggcagttggcaggatatttatatacgatattaatacttttgctactggcacactaaaatttatttgcccgtaaggggacgtccttcggtggttatataaataatcccgtagggggagggggatgtcccgtagggggaggggagtggaggctccaacggaggttggagcttctttggtttcctaggcattatttaaatattttttaaccctagcactagaactgagattccagacggcgacccgtaaagttcttcagtcccctcagctttttcacaaccaagttcgggatggattggtgtgggtccaactgagcaaagagcaccaaggttaactgcatctctgtgagatgctagttaaactaagcttagcttagctcataaacgatagttacccgcaaggggttatgtaattatattataaggtcaaaatcaaacggcctttagtatatctcggctaaagccattgctgactgtacacctgatacctatataacggcttgtctagccgcggccttagagagcactcatcttgagtttagcttcctacttagatgctttcagcagttatctatccatgcgtagctacccagcgtttcccattggaatgagaactggtacacaattggcatgtcctttcaggtcctctcgtactatgaaaggctactctcaatgctctaacgcctacaccggatatggaccaaactgtctcacgcatgaaattttaaagccgaataaaacttgcggtctttaaaactaacccctttactttcgtaaaggcatggactatgtcttcatcctgctactgttaatggcaggagtcggcgtattatactttcccactctcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 11 - protein, 338 aa, Pseudomonas stutzeri
MLPKLVITHRVHDEILQLLAPHCELMTNQTDSTLTREEILRRCRDAQAMMAFMPDRVDADFLQACPELRVVGCALKGFDNFDVDACTARGVWLTFVPDLLTVPTAELAIGLAVGLGRHLRAADAFVRSGEFQGWQPQFYGTGLDNATVGILGMGAIGLAMADRLQGWGATLQYHEAKALDTQTEQRLGLRQVACSELFASSDFILLALPLNADTQHLVNAELLALVRPGALLVNPCRGSVVDEAAVLAALERGQLGGYAADVFEMEDWARADRPRLIDPALLAHPNTLFTPHIGSAVRAVRLEIERCAAQNIIQVLAGARPINAANRLPKAEPAACEF

SEQ ID NO: 12 - DNA, 1017 bp, Artificial sequence
  Codon optimized sequence of PTXD from Pseudomonas stutzeri
atgctgccgaagctggtcatcacccaccgcgtccacgacgagatcctgcagctgctggccccgcactgcgagctgatgacgaaccagaccgactcgaccctgacgcgcgaggagatcctgcgccgctgccgcgacgcgcaggctatgatggccttcatgccggaccgcgtggacgctgacttcctgcaggcttgcccggagctgcgcgtggtcggctgcgctctgaagggcttcgacaacttcgacgtggacgcttgcaccgctcgcggcgtgtggctgacgttcgtcccggacctgctgaccgtgccgacggctgagctggccatcggcctggctgtcggcctgggccgccacctgcgcgccgcggacgctttcgtgcgctccggcgagttccagggctggcagccgcagttctacggcaccggcctggacaacgctacggtcggcatcctgggcatgggcgctatcggcctggctatggctgaccgcctgcagggctggggcgctaccctgcagtaccacgaggctaaggccctggacacccagacggagcagcgcctgggcctgcgccaggtggcttgcagcgagctgttcgcctcgtccgacttcatcctgctggctctgccgctgaacgctgacacccagcacctggtcaacgctgagctgctggctctggtgcgccccggcgctctgctggtcaacccgtgccgcggctctgtggtggacgaggctgccgtgctggctgctctggagcgcggccagctgggcggctacgccgcggacgtcttcgagatggaggactgggcgcgcgctgaccgcccgcgcctgatcgacccggctctgctggctcacccgaacaccctgttcacgccgcacatcggcagcgccgtgcgcgcggtccgcctggagatcgagcgctgcgctgcccagaacatcatccaggtgctggccggcgcccgcccgatcaacgctgccaaccgcctgccgaaggctgagccggctgcttgcgaattctaa

SEQ ID NO: 13 - DNA, 4657 bp, Artificial sequence
  Construct for the expression of PTXD
tcgctgaggcttgacatgattggtgcgtatgtttgtatgaagctacaggactgatttggcgggctatgagggcgggggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcagctagcttaagatcccatcaagcttgcatgccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaaatggccaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggatggccatgcatatggccaagctgaccagcgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggacgccccggtgaagcagaccctgaacttcgacctgctgaagctggcgggcgacgtggagagcaacccgggccccgaattcatgctgccgaagctggtcatcacccaccgcgtccacgacgagatcctgcagctgctggccccgcactgcgagctgatgacgaaccagaccgactcgaccctgacgcgcgaggagatcctgcgccgctgccgcgacgcgcaggctatgatggccttcatgccggaccgcgtggacgctgacttcctgcaggcttgcccggagctgcgcgtggtcggctgcgctctgaagggcttcgacaacttcgacgtggacgcttgcaccgctcgcggcgtgtggctgacgttcgtcccggacctgctgaccgtgccgacggctgagctggccatcggcctggctgtcggcctgggccgccacctgcgcgccgcggacgctttcgtgcgctccggcgagttccagggctggcagccgcagttctacggcaccggcctggacaacgctacggtcggcatcctgggcatgggcgctatcggcctggctatggctgaccgcctgcagggctggggcgctaccctgcagtaccacgaggctaaggccctggacacccagacggagcagcgcctgggcctgcgccaggtggcttgcagcgagctgttcgcctcgtccgacttcatcctgctggctctgccgctgaacgctgacacccagcacctggtcaacgctgagctgctggctctggtgcgccccggcgctctgctggtcaacccgtgccgcggctctgtggtggacgaggctgccgtgctggctgctctggagcgcggccagctgggcggctacgccgcggacgtcttcgagatggaggactgggcgcgcgctgaccgcccgcgcctgatcgacccggctctgctggctcacccgaacaccctgttcacgccgcacatcggcagcgccgtgcgcgcggtccgcctggagatcgagcgctgcgctgcccagaacatcatccaggtgctggccggcgcccgcccgatcaacgctgccaaccgcctgccgaaggctgagccggctgcttgcgaattctaactcgagcaccaccaccaccaccacggcaagcccatccccaaccccctgctgggcctggacagcaccgagaacctgtacttccagggggtaccaggatccggaagatctggtctagagggcaagcccatccccaaccccctgctgggcctggacagcacccgtaccggtcaccaccaccaccaccactaactgcagccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagcggcggtatttcacaccgcatcaggtggcacttttcggggaaatgtgcgcggaacccctatttgtttatttttctaaatacattcaaatatgtatccgctcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgttgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgg

SEQ ID NO: 14 - protein, 462 aa, Thermus thermophilus
MLARRSFLQAAAGSLVLGLARAQGPSFPEPKVVRSQGGLLSLKLSATPTPLALAGQRATLLTYGGSFPGPTLRVRPRDTVRLTLENRLPEPTNLHWHGLPISPKVDDPFLEIPPGESWTYEFTVPKELAGTFWYHPHLHGRVAPQLFAGLLGALVVESSLDAIPELREAEEHLLVLKDLALQGGRPAPHTPMDWMNGKEGDLVLVNGALRPTLVAQKATLRLRLLNASNARYYRLALQDHPLYLIAADGGFLEEPLEVSELLLAPGERAEVLVRLRKEGRFLLQALPYDRGAMGMMDMGGMAHAMPQGPSRPETLLYLIAPKNPKPLPLPKALSPFPTLPAPVVTRRLVLTEDMMAARFFINGQVFDHRRVDLKGQAQTVEVWEVENQGDMDHPFHLHVHPFQVLSVGGRPFPYRAWKDVVNLKAGEVARLLVPLREKGRTVFHCHIVEHEDRGMMGVLEVG

SEQ ID NO: 15 - DNA, 1400 bp, Artificial sequence
  Codon usage optimized nucleotidic sequence encoding for laccase of Thermus thermophilus
ccatggctttagctcgtcgttcatttttacaagctgctgctggttcattagttttaggtttagctcgtgctcaaggtccatcatttccagaaccaaaagttgttcgttcacaaggtggtttattatcattaaaattatcagctacaccaacaccattagctttagctggtcaacgtgctacattattaacatatggtggttcatttccaggtccaacattacgtgttcgtccacgtgatacagttcgtttaacattagaaaatcgtttaccagaaccaacaaatttacattggcatggtttaccaatttcaccaaaagttgatgatccatttttagaaattccaccaggtgaatcatggacatatgaatttacagttccaaaagaattagctggtacattttggtatcatccacatttacatggtcgtgttgctccacaattatttgctggtttattaggtgctttagttgttgaatcatcattagatgctattccagaattacgtgaagctgaagaacatttattagttttaaaagatttagctttacaaggtggtcgtccagctccacatacaccaatggattggatgaatggtaaagaaggtgatttagttttagttaatggtgctttacgtccaacattagttgctcaaaaagctacattacgtttacgtttattaaatgcttcaaatgctcgttattatcgtttagctttacaagatcatccattatatttaattgctgctgatggtggttttttagaagaaccattagaagtttcagaattattattagctccaggtgaacgtgctgaagttttagttcgtttacgtaaagaaggtcgttttttattacaagcattaccatatgatcgtggtgctatgggtatgatggatatgggtggtatggctcatgctatgccacaaggtccatcacgtccagaaacattattatatttaattgctccaaaaaatccaaaaccattaccattaccaaaagcattatcaccatttccaacattaccagctccagttgttacacgtcgtttagttttaacagaagatatgatggctgctcgtttttttattaatggtcaagtttttgatcatcgtcgtgttgatttaaaaggtcaagctcaaacagttgaagtttgggaagttgaaaatcaaggtgatatggatcatccatttcatttacatgttcatccatttcaagttttatcagttggtggtcgtccatttccatatcgtgcttggaaagatgttgttaatttaaaagctggtgaagttgctcgtttattagttccattacgtgaaaaaggtcgtacagtttttcattgtcatattgttgaacatgaagatcgtggtatgatgggtgttttagaagttggttaagcatgc

SEQ ID NO: 16 - protein, 242 aa, Thermus thermophilus
MTLLRTPLPVPHGFTTREGGVSEGPFRSLNLSAATGDDPERVAENQRRVLAAFGHPPVAGLRQVHGTEVHPVEGPGLWEGDGLLTRTPGLLLRVGVADCYPLLLYHPKGAVGALHAGWRGVVGGILPKALERLEAVYRLDPTEVHLAIGPGIGGACYQVGEEVVARFAEAGLFTFREDPAAPGKYLLDLEKALLLQARRAGLREERIYRVGLCTHCAPNLFSHRRDRGRTGRMWGLVMLPPR

SEQ ID NO: 17 - DNA, 740 bp, Artificial sequence
  Codon usage optimized nucleotidic sequence of polyphenol oxidase from Thermus thermophilus
ccatggctacattattacgtacaccattaccagttccacatggttttacaacacgtgaaggtggtgtttcagaaggtccatttcgttcattaaatttatcagctgctacaggtgatgatccagaacgtgttgctgaaaatcaacgtcgtgttttagctgcttttggtcatccaccagttgctggtttacgtcaagttcatggtacagaagttcatccagttgaaggtccaggtttatgggaaggtgatggtttattaacacgtacaccaggtttattattacgtgttggtgttgctgattgttatccattattattatatcatccaaaaggtgctgttggtgctttacatgctggttggcgtggtgttgttggtggtattttaccaaaagcattagaacgtttagaagctgtttatcgtttagatccaacagaagttcatttagctattggtccaggtattggtggtgcttgttatcaagttggtgaagaagttgttgctcgttttgctgaagctggtttatttacatttcgtgaagatccagctgctccaggtaaatatttattagatttagaaaaagcattattattacaagctcgtcgtgctggtttacgtgaagaacgtatttatcgtgttggtttatgtacacattgtgctccaaatttattttcacatcgtcgtgatcgtggtcgtacaggtcgtatgtggggtttagttatgttaccaccacgttaagcatgc

(19) United States
(12) Patent Application Publication
(10) Pub. No.: US 2021/0395702 A1
(43) Pub. Date: Dec. 23, 2021

(54) TRANSGENIC MICROALGAE FOR THE PRODUCTION OF PLANT CELL WALL DEGRADING ENZYMES HAVING HEAT-STABLE CELLULOLYTIC ACTIVITY

(71) Applicants: Roberto BASSI, Verona (IT); Luca DALL'OSTO, San Martino Buon Albergo (VR) (IT); Manuel BENEDETTI, Civitavecchia (RM) (IT)

(72) Inventors: Roberto BASSI, Verona (IT); Luca DALL'OSTO, San Martino Buon Albergo (VR) (IT); Manuel BENEDETTI, Civitavecchia (RM) (IT)

(21) Appl. No.: 17/288,559

(22) PCT Filed: Jul. 30, 2019
(86) PCT No.: PCT/IB2019/056477

§ 371 Date: Apr. 25, 2021

(30) Foreign Application Priority Data
Oct. 29, 2018 (IT) 102018000009867

(51) Int. Cl. C12N 9/02; C12N 9/42; C12N 9/24; C12N 1/12; C12N 15/82; C12N 9/98; C12P 19/14

(52) U.S. Cl. CPC C12N 9/0004; C12P 19/14; C12N 9/2445; C12N 9/2482; C12N 9/0061; C12N 9/0057; C12Y 110/03002; C12Y 120/01001; C12Y 302/01004; C12Y 302/01008; C12Y 302/01021; C12N 1/12; C12N 15/8214; C12N 9/98; C12N 9/2437

(57) ABSTRACT

The present invention relates to transgenic microalgae for the production of cell wall degradative enzymes having a heat-stable cellulolytic activity (HCWDEs) and their relative uses in the biodegradation of cellulose or lignocellulose sources in the industrial field.

The present invention relates to transgenic microalgae for the production of plant cell wall degradative enzymes having a heat-stable cellulolytic activity (HCWDEs) and relative uses in the biodegradation of cellulose or lignocellulose sources in the industrial field.

Lignocellulose is the most abundant source of organic carbon on earth and is a reservoir of carbohydrates with a high potential for use in the production of biofuels. Unfortunately, its extremely recalcitrant nature to conversion into simple sugars enormously limits its exploitation in this area (Sanderson K., 2011; Saini J. K. et al. 2015).

Methods that include physical, physico-chemical, chemical and biological treatments are commonly used for reducing recalcitrance to hydrolysis and promoting saccharification (Harmsen P F H et al. 2010; Badiei M. et al. 2014; Kumar A K and Sharma S., 2017).

Chemical treatment, however, is harmful to the environment and is in contrast with the idea of using lignocellulose for producing a form of sustainable energy; furthermore, this kind of treatment generates reaction derivatives that inhibit the microbial metabolism, invalidating the conversion of simple sugars into bioenergetic products such as ethanol, lipids and methane (Jönsson L. J. and Martin C., 2016).

Conversely, although physical methods are relatively non-polluting, their large-scale application is expensive (Kumar A. K. and Sharma S., 2017).

Biological treatment involves the use of plant wall degradative enzymes (CWDEs) of a microbial nature, which, more often than not, are obtained by cultivating mesophilic molds and bacteria with lignocellulolytic activities (Sanchez C., 2009). Generally speaking, these microorganisms secrete a wide range of CWDEs but in small quantities, as they are required for their strict requirements.

To date, the real biotechnological challenge consists in selecting strains capable of expressing large quantities of CWDEs, which can then be exploited in the degradation of lignocellulose.

The most promising strains should be characterized by the following features:

(i) ability to express the selected enzyme

(ii) high productivity (referring to the amount of enzyme produced in the unit of time) and

(iii) low production cost.

An organism that is characterized by these phenotypic traits is certainly a valid candidate for the large-scale production of CWDEs.

From this point of view, microalgae can be promising biofactories, as they are characterized by a high growth rate and very low production costs (Brasil B. et al., 2017).

There are, however, limitations in the use of microalgae as recombinant protein biofactories; first of all, a limited knowledge of microalga as a heterologous expression system. The nuclear expression of bacterial and fungal CWDE-coding transgenes, for example, has already been attempted in the alga model Chlamydomonas reinhardtii; the expression yield was below expectations (Rasala B. A. et al. 2012) even if the existence of an endogenous cellulolytic system led the opposite to be assumed (Blifernez-Klassen et al., 2012).

Among the various factors that negatively influence the expression of transgenes in microlage, gene silencing plays a predominant role (Schroda M., 2006).

In order to avoid this problem, the authors of the present invention tried to express for the first time a set of CWDEs with a heat-stable activity (HCWDEs) in the chloroplast of the microalga. The success of this approach was difficult to predict as most of the carbohydrate metabolism is localized in the chloroplast and therefore the expression of cellulases could theoretically interfere with this metabolism.

As they are of a bacterial origin, HCWDEs (abbreviation HCs) do not require post-translational modifications for their correct functioning and consequently, the prokaryotic nature of the chloroplast is congenial for this purpose.

The degradation of plant biomass through the use of heat-stable HC enzymes has various advantages compared to that using their enzymatic counterparts with a thermolabile activity (Anitori R. P., 2012; Peng X. et al., 2015). The high temperature at which HCs exert their activity promotes the partial detachment of lignin from the cellulose fibers, favouring the activity of the HCs. and at the same time prevents contamination by mesophilic microbes (Sarmiento F. et al., 2015).

Furthermore, CWDE protein inhibitors and plant defense proteases are inactivated by high temperatures and as a result, any possible inhibitory mechanisms arising from these defense proteins cannot occur.

The robust structure of HCs, on the contrary, gives a marked enzymatic stability, even in the presence of aggressive chemical reagents, ionic detergents and extreme pH conditions which, in turn, can promote the weakening of lignocellulose, further increasing the efficiency of the enzymatic hydrolysis reactions.

As a further step towards sustainability, the microalgae expressing HCs in the chloroplast (hereinafter referred to as HC-algae) were also engineered to express the phosphite dehydrogenase D (PTXD) of P. stutzeri in the cytoplasm, whose expression gives the microalgae the capacity of using the phosphite ion as the sole source of phosphorous, thus allowing the cultivation of the alga in growth media containing phosphite ion, instead of phosphate ion (Costas A M G et al., 2001; Loera-Quezada M M et al., 2016). The double-transgenic microalgae (hereinafter referred to as HC-PTXD algae) can be cultivated in this type of soil without the need for using sterile materials and procedures as the phosphite ion has an antifungal action and cannot be metabolized by most common bacteria.

It should be noted that sterilization materials and procedures have a great impact on the cost of microalgae cultivation and in fact, in some cases, it can represent up to 50% of the final production cost. Referring to the prices of some microalgae-producing companies, their cultivation under conditions of non-sterility would lower their production costs up to €5 kg⁻¹ D W (Rodolfi L. et al., 2009); considering that the products currently available on the market based on bacterial powders with thermolabile cellulolytic activities have costs that are around 30-40 € kg⁻¹, it is clear that products based on microalgae transformed with heat-stable enzymes could prove to be very competitive.

An object of the present invention therefore relates to a combination of transgenic microalgae in which each transgenic microalga expresses a phosphite dehydrogenase D of bacterial origin and a heat-stable plant cell wall degradative enzyme selected from the group consisting of endoglucanase B of Thermotoga neapolitana (SEQ ID NR: 1), the portion with a cellobiohydrolase activity of the cellulosome CelB of Caldicellulosiruptor saccharolyticus (SEQ ID NR: 3) and the beta-glucosidase of Pyrococcus furiosus (SEQ ID NR: 5), wherein said endoglucanase B of Thermotoga neapolitana is encoded by the nucleotide sequence with codon usage optimized for chloroplast expression (SEQ ID Nr: 2), said portion of the cellulosome CelB of Caldicellulosiruptor saccharolyticus is encoded by the nucleotide sequence with codon usage optimized for chloroplast expression (SEQ ID Nr: 4), and said beta-glucosidase of Pyrococcus furiosus is encoded by the nucleotide sequence with codon usage optimized for chloroplast expression (SEQ ID Nr: 6).

Optionally, a transgenic microalga expressing a phosphite dehydrogenase D of bacterial origin and xylanase XynA of Thermotoga neapolitana (SEQ ID NR: 7) can be added to the above-mentioned combination of transgenic microalgae.

The above-mentioned xylanase XynA of Thermotoga neapolitana is preferably encoded by the nucleotide sequence with codon usage optimized for the chloroplast expression SEQ ID Nr: 8.

According to an alternative embodiment of the present invention, a transgenic microalga expressing a phosphite dehydrogenase D of bacterial origin and a ligninase (in addition to or in place of xylanase), preferably selected from laccase of Thermus thermophilus (SEQ ID NR: 14) and polyphenol oxidase of Thermus thermophilus (SEQ ID NR 16), can be added to the combination.

Again according to a preferred embodiment of the invention, said heat-stable plant cell wall degradative enzyme is selected from the group consisting of:
endoglucanase B CelB of Thermotoga neapolitana (T-EG) having the amino acid sequence:

(SEQ ID NO: 1)
MAEVVLTDIGATDITFKGFPVTMELNFWNVKSYEGETWLKFDGEKVQFYA
DILYNIVLQNPDSWVHGYPEIYYGYKPWAAHNSGTEILPVKVKDLPDFYV
TLDYSIWYENDLPINLAMETWITRKPDQTSVSSGDVEIMVWFYNNILMPG
GQKVDEFTTTIEINGSPVEIKWDVYFAPWGWDYLAFRLTTPMKDGRVKFN
VKDFVEKAAEVIKKHSTRVENFDEMYFCVWEIGTEFGDPNTTAAKFGWTF
KDFSVEIGEYPYDVPDYA
with HA tail

portion with a cellobiohydrolase activity of the cellulosome CelB of Caldicellulosiruptor saccharolyticus (C-CBH) having the amino acid sequence:

(SEQ ID NO: 3)
MGVTTSSPTPTPTPTVTVTPTPTPTPTPTVTATPTPTPTPVSTPATGGQI
KVLYANKETNSTTNTIRPWLKVVNSGSSSIDLSRVTIRYWTTVDGERAQS
AVSDWAQIGASNVTFKFVKLSSSVSGADYYLEIGFKSGAGQLQPGKDTGE
IQIRFNKSDWSNYNQGNDWSWLQSMTSYGENEKVTAYIDGVLVWGQEPSG
ATPAPTMTVAPTATPTPTLSPTVTPTPAPTQTAIPTPTLTPNPTPTSSIP
DDTNDDWLYVSGNKIVDKDGRPVWLTGINWFGYNTGTNVFDGVWSCNLKD
TLAEIANRGFNLLRVPISAELILNWSQGIYPKPNINYYVNPELEGKNSLE
VFDIVVQTCKEVGLKIMLDIHSIKTDAMGHIYPVWYDEKFTPEDFYKACE
WITNRYKNDDTIIAFDLKNEPHGKPWQDTTFAKWDNSTDINNWKYAAETC
AKRILNINPNLLIVIEGIEAYPKDDVTWTSKSSSDYYSTWWGGNLRGVRK
YPINLGKYQNKVVTSPHDYGPSVYQQPWFYPGFTKESLLQDCWRPNWAYI
MEENIAPLLIGEWGGHLDGADNEKWMKYLRDYIIENHIHHTFWCFNANSG
DTGGLVGYDFTTWDEKKYSFLKPALWQDSQGRFVGLDHKRPLGTNGKNIN
ITTYYNNNEPEPVPASKYPYDVPDYA
with HA tail

beta-glucosidase of Pyrococcus furiosus (P-BG) having the amino acid sequence:

(SEQ ID NO: 5)
MAKFPKNFMFGYSWSGFQFEMGLPGSEVESDWWVWVHDKENIASGLVSGD
LPENGPAYWHLYKQDHDIAEKLGMDCIRGGIEWARIFPKPTFDVKVDVEK
DEEGNIISVDVPESTIKELEKIANMEALEHYRKIYSDWKERGKTFILNLY
HWMPLWIHDPIAVRKLGPDRAPAGWLDEKTVVEFVKFAAFVAYHLDDLVD
MWSTMNEPNVVYNQGYINLRSGFPPGYLSFEAAEKAKFNLIQAHIGAYDA
IKEYSEKSVGVIYAFAWHDPLAEEYKDEVEEIRKKDYEFVTILHSKGKLD
WIGVNYYSRLNYGAKDGHLVPLPGYGFMSERGGFAKSGRPASDFGWEMYP
EGLENLLKYLNNAYELPMIITENGMADAADRYRPHYLVSHLKAVYNAMKE
GADVRGYLHWSLTDNYEWAQGFRMRFGLVYVDFETKKRYLRPSALVFREI
ATQKEIPEELAHLADLKFVTRKYPYDVPDYA
with HA tail

optionally, xylanase XynA of Thermotoga neapolitana (T-XY) having the amino acid sequence:

(SEQ ID NO: 7)
MATGALGFGGKGVSPFETVLVLSFEGNTDGASPFGKDVVVTASQDVAADG
EYSLKVENRTSVWDGVEIDLTGKVNTGTDYLLSFHVYQTSDSPQLFSVLA
RTEDEKGERYKILADKVVVPNYWKEILVPFSPTFEGTPAKFSLIITSPKK
TDFVFYVDNVQVLTPKEAGPKVVYETSFEKGIGDWQPRGSDVKISISPKV
AHSGKKSLFVSNRQKGWHGAQISLKGILKTGKTYAFEAWVYQESGQDQTI
IMTMQRKYSSDSSTKYEWIKAATVPSGQWVQLSGTYTIPAGVTVEDLTLY
FESQNPTLEFYVDDVKWDTTSAEIKLEMNPEEEIPALKDVLKDYFRVGVA
LPSKVFINQKDIALISKHFNSITAENEMKPDSLLAGIENGKLKFRFETAD
KYIEFAQQNGMVVRGHTLVWHNQTPEWFFKDENGNLLSKEEMTERLREYI
HTVVGHFKGKVYAWDVVNEAVDPNQPDGLRRSTWYQIMGPDYIELAFKFA
READPNAKLFYNDYNTFEPKKRDIIYNLVKSLKEKGLIDGIGMQCHISLA
TDIRQIEEAIKKFSTIPGIEIHITELDISVYRDSTSNYSEAPRTALIEQA
HKMAQLFKIFKKYSNVITNVTFWGLKDDYSWRATRRNDWPLIFDKDYQAK
LAYWAIVAPEVLPPLPKESKISEGEAVVVGMMDDSYMMSKPIEIYDEEGN
VKATIRAIWKDSTIYVYGEVQDATKKPAEDGVAIFINPNNERTPYLQPDD
TYVVLWTNWKSEVNREDVEVKKFVGPGFRRYSFEMSITIPGVEFKKDSYI
GFDVAVIDDGKWYSWSDTTNSQKTNTMNYGTLKLEGVMVATAKYGTPVID
GEIDDIWNTTEEIETKSVAMGSLEKNATAKVRVLWDEENLYVLAIVKDPV
LNKDNSNPWEQDSVEIFIDENNHKTGYYEDDDAQFRVNYMNEQSFGTGAS
AARFKTAVKLIEGGYIVEAAIKWKTIKPSPNTVIGFNVQVNDANEKGQRV
GIISWSDPTNNSWRDPSKFGNLRLIKYPYDVPPYA
with HA tail

optionally, laccase of Thermus thermophilus having the amino acid sequence:

(SEQ ID NO: 14)
MLARRSFLQAAAGSLVLGLARAQGPSFPEPKVVRSQGGLLSLKLSATPTP
LALAGQRATLLTYGGSFPGPTLRVRPRDTVRLTLENRLPEPTNLHWHGLP
ISPKVDDPFLEIPPGESWTYEFTVPKELAGTFWYHPHLHGRVAPQLFAGL
LGALVVESSLDAIPELREAEEHLLVLKDLALQGGRPAPHTPMDWMNGKEG
DLVLNGALRPTLVAQKATLRLRLLNASNARYYRLALQDHPLYLIAADGGF
LEEPLEVSELLLAPGERAEVLVRLRKEGRFLLQALPYDRGAMGMMDMGGM
AHAMPQGPSRPETLLYLIAPKNPKPLPLPKALSPFPTLPAPVVTRRLAVL
TEDMMAARFFINGQVFDHRRVDLKGQAQTVEVWEVENQGDMDHPFHLHVH
PFQVLSVGGRPFPYRAWKDVVNLKAGEVARLLVPLREKGRTVFHCHIVEH
EDRGMMGVLEVG

optionally, polyphenol oxidase of Thermus thermophilus having the amino acid sequence:

(SEQ ID NO: 16)
MTLLRTPLPVPHGFTTREGGVSHGPFRSLNLSAATGDDPERVAENQRRVL
AAFGHPPVAGLRQVHGTEVHPVEGPGLWEGDGLLTRTTGLLLRVGVADCY
PLLLYHPKGAVGALHAGWRGVVGGILPKALERLEAVYRLDPTEVHLAIGP
GIGGACYQVGEEVVARFAEAGLFTREDPAAPGKYLLDLEKALLLQARRAG
LREERIYRVGLCTHCAPNLFSHRRDRGRTGRMWGLVMLPPR.

The above nucleotide sequence of endoglucanase B of Thermotoga neapolitana is encoded by the nucleotide sequence with codon usage optimized for the chloroplast expression SEQ ID NR: 2; the above-mentioned portion CBM3GH5 of the CelB cellulosome of Caldicellulosiruptor saccharolyticus is encoded by the nucleotide sequence with codon usage optimized for the chloroplast expression SEQ ID NR: 4; the above-mentioned beta-glucosidase of Pyrococcus furiosus is encoded by the nucleotide sequence with codon usage optimized for the chloroplast expression SEQ ID NR: 6; the above-mentioned xylanase XynA of Thermotoga neapolitana is encoded by the nucleotide sequence with codon usage optimized for the chloroplast expression SEQ ID NR: 8; the above-mentioned laccase of Thermus thermophilus is encoded by the nucleotide sequence with codon usage optimized for the chloroplast expression SEQ ID NR: 15 and the above-mentioned polyphenol oxidase of Thermus thermophilus is encoded by the nucleotide sequence with codon usage optimized for chloroplast expression SEQ ID NR: 17.

According to a preferred embodiment of the present invention, the transgenic microalgae belong to the Chlamydomonas reinhardtii species.

In a preferred embodiment of the present invention said phosphite dehydrogenase D comes from Pseudomonas stutzeri (PTXD) and has the following amino acid sequence:

(SEQ ID NO: 11)
MLPKLVITHRVHDEILQLLAPHCELMTNQTDSTLTREEILRRCRDAQAMM
AFMPDRVDADFLQACPELRVVGCALKGFDNFDVDACTARGVWLTFVPDLL
TVPTAELAIGLAVGLGRHLRAADAFVRSGEFQGWQPQFYGTGLDNATVGI
LGMGAIGLAMADRLQGWGATLQYHEAKALDTQTEQRLGLRQVACSELFAS
SDFILLALPLNADTQHLVNAELLALVRPGALLVNPCRGSVVDEAAVLAAL
ERGQLGGYAADVFEMEDWARADRPRLIDPALLAHPNTLFTPHIGSAVRAV
RLEIERCAAQNIIQVLAGARPINAANRLPKAEPAACEF

The above-mentioned amino acid sequence of the phosphite dehydrogenase D of Pseudomonas stutzeri (PTXD) is encoded by the following optimized nucleotide sequence:

(SEQ ID NO: 12)
ATGCTGCCGAAGCTGGTCATCACCCACCGCGTCCACGACGAGATCCTGCA
GCTGCTGGCCCCGCACTGCGAGCTGATGACGAACCAGACCGACTCGACCC
TGACGCGCGAGGAGATCCTGCGCCGCTGCCGCGACGCGCAGGCTATGATG
GCCTTCATGCCGGACCGCGTGGACGCTGACTTCCTGCAGGCTTGCCCGGA
GCTGCGCGTGGTCGGCTGCGCTCTGAAGGGCTTCGACAACTTCGACGTGG
ACGCTTGCACCGCTCGCGGCGTGTGGCTGACGTTCGTCCCGGACCTGCTG
ACCGTGCCGACGGCTGAGCTGGCCATCGGCCTGGCTGTCGGCCTGGGCCG
CCACCTGCGCGCCGCGGACGCTTTCGTGCGCTCCGGCGAGTTCCAGGGCT
GGCAGCCGCAGTTCTACGGCACCGGCCTGGACAACGCTACGGTCGGCATC
CTGGGCATGGGCGCTATCGGCCTGGCTATGGCTGACCGCCTGCAGGGCTG
GGGCGCTACCCTGCAGTACCACGAGGCTAAGGCCCTGGACACCCAGACGG
AGCAGCGCCTGGGCCTGCGCCAGGTGGCTTGCAGCGAGCTGTTCGCCTCG
TCCGACTTCATCCTGCTGGCTCTGCCGCTGAACGCTGACACCCAGCACCT
GGTCAACGCTGAGCTGCTGGCTCTGGTGCGCCCCGGCGCTCTGCTGGTCA
ACCCGTGCCGCGGCTCTGTGGTGGACGAGGCTGCCGTGCTGGCTGCTCTG
GAGCGCGGCCAGCTGGGCGGCTACGCCGCGGACGTCTTCGAGATGGAGGA
CTGGGCGCGCGCTGACCGCCCGCGCCTGATCGACCCGGCTCTGCMGCTCA
CCCGAACACCCTGTTCACGCCGCACATCGGCAGCGCCGTGCGCGCGGTCC
GCCTGGAGATCGAGCGCTGCGCTGCCCAGAACATCATCCAGGTGCTGGCC
GGCGCCCGCCCGATCAACGCMCCAACCGCCTGCCGAAGGCTGAGCCGGCT
GCTTGCGAATTCTAA.

The present invention also relates to the use of a combination of transgenic microalgae as defined above, for the production of a mixture of heat-stable plant cell wall degradative enzymes in a culture medium containing the phosphite ion as a source of phosphorus.

The present invention also relates to a process for the production of a mixture of heat-stable plant cell wall degradative enzymes, comprising the following steps:
a) cultivation of a combination of transgenic microalgae as defined above in a culture medium comprising the phosphite ion as sole phosphorous source, in a photobioreactor;
b) drying the microalgae, preferably by means of freeze-drying, at −80° C.;
c) extraction of the enzymes alternatively by means of sonication, short heat treatment at 80° C., non-denaturing conditions or denaturing conditions.

The possibility of growing the microalgae in high volumes thanks to the use of the phosphite ion makes it possible to avoid using sterilization processes which, on a large scale, would be economically unsustainable. By way of example, the mixture of heat-stable plant cell wall degradative enzymes produced by the combination of transgenic microalgae according to the present invention can be advantageously used in biogas production plants for degrading the cellulosic substrate into the corresponding constituent monosaccharides. This is effected in bioreactors at 30-40° C., where the bacteria present produce methane. The lyophilized powder comprising heat-stable cellulolytic enzymes can be used directly on the substrate in quantities ranging from 1 Kg:1 ton of substrate to 5 Kg:1 ton of substrate, in appropriate treatment tanks at temperatures ranging from 70 to 100° C. before entering the bioreactor.

The present invention also relates to a mixture of heat-stable plant cell wall degradative enzymes that can be obtained according to the process described above, characterized in that it comprises endoglucanase B of Thermotoga neapolitana (SEQ ID NR 1), the portion with a cellobiohydrolase activity of the cellulosome CelB of Caldicellulosiruptor saccharolyticus (SEQ ID NR 3), the beta-glucosidase of Pyrococcus furiosus (SEQ ID NR: 5), in a ratio [g strain endoglucanase B:gstrain cellobiohydrolase portion of the cellulosome CelB:gstrain beta-glucosidase] of 20:50:30, which corresponds to a molar enzymatic ratio [mol. endoglucanase B:mol. cellobiohydrolase portion of the cellulosome CelB:mol. beta-glucosidase] of 5:65:30. This mixture is characterized by a specific activity (Enzyme units per g of dry weight of algal mixture) ranging from 10 to 30 U towards the CMC substrate and from 8 to 24 U towards the pNPG substrate.

In a further particularly preferred embodiment, the mixture of heat-stable plant cell wall degradative enzymes that can be obtained according to the process described above, is characterized in that it comprises endoglucanase B of Thermotoga neapolitana (SEQ ID NR: 1), the portion with a cellobiohydrolase activity of the cellulosome CelB of Caldicellulosiruptor saccharolyticus (SEQ ID NR: 3), the beta-glucosidase of Pyrococcus furiosus (SEQ ID NR: 5), and xylanase XynA of Thermotoga neapolitana (SEQ ID NR:7), in a ratio [g strain endoglucanase B:gstrain cellobiohydrolase portion of the cellulosome CelB:gstrain beta-glucosidase:gstrain xylanase] of 20:40:20:20. The addition of xylanase XynA to the above ternary mixture results in a molar enzymatic ratio [mol. endoglucanase B:mol. cellobiohydrolase portion of the cellulosome CelB:mol. beta-glucosidase: mol. xylanase] equal to 5:60:25:10. In this case, the specific activity ranges from 8 to 24 U towards the CMC substrate, from 6 to 18 U towards the pNPG substrate and from 1 to 3 U towards the xylan substrate.

The mixtures of heat-stable plant cell wall degradative enzymes according to the invention defined above can be further characterized in that they comprise in addition or alternatively (to xylanase), a ligninase selected from laccase of Thermus thermophilus (SEQ ID NR: 14) and polyphenol oxidase of Thermus thermophilus (SEQ ID NR: 16).

According to a particularly preferred embodiment, the mixture of heat-stable plant cell wall degradative enzymes of the invention is in the form of a lyophilized powder.

Finally, the invention relates to the use of xylanase XynA of Thermotoga neapolitana (SEQ ID NR: 7) in mixture with heat-stable plant cell wall degradable enzymes of the hemicellulase type comprising endoglucanase B of Thermotoga neapolitana (SEQ ID NR: 1), the portion with a cellobiohydrolase activity of the cellulosome CelB of Caldicellulosiruptor saccharolyticus (SEQ ID NR: 3) and beta-glucosidase of Pyrococcus furiosus (SEQ ID NR: 5) as a preventive treatment for the biodegradation of lignocellulose-based substrates.

A further object of the present invention relates to the use of the mixtures of heat-stable plant cell wall degradative enzymes defined above for the biodegradation of cellulose-based substrates (i.e. paper pulp) or lignocellulose (for example, in the production of biofuels). In the latter case, it is obviously necessary to provide for the inclusion of microalgae in the mixture, a microalga transformed to express enzymes of the ligninase type.

The present invention will now be described for illustrative, but non-limiting, purposes, according to a preferred embodiment with particular reference to the attached figures, in which:

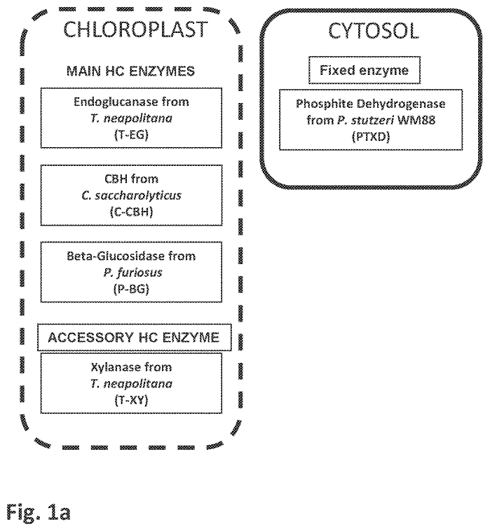

FIG. 1 shows a graphical representation of the C. reinhardtii cell as a biofactory of HC enzymes. a) List of enzymes that make up the cellulolytic machinery [T-EG=Endoglucanase, C-CBH=Cellobiohydrolase, P-BG=beta-glucosidase, T-XY=Xylanase]. b) Each strain co-expresses the phosphite dehydrogenase enzyme (PTXD) and one of the HC enzymes shown in a).

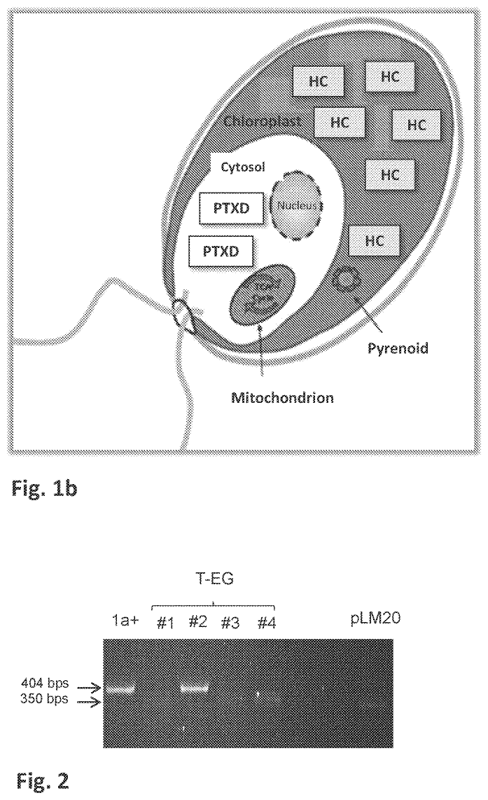

FIG. 2 shows the results of the evaluation of the homoplasmic condition in HC-algae. The amplicon of 404 and 350 bp indicates the presence of the inverted-repeated region (IR) of the wild-type (WT) and recombinant chloroplast plasmid, respectively. The DNA of the strain 1a+ and the plasmid pLM20 were used as a negative and positive control of the homoplasmic condition, respectively. The analysis of four transformants of the alga expressing T-EG (#1-4) are shown as a representative result.

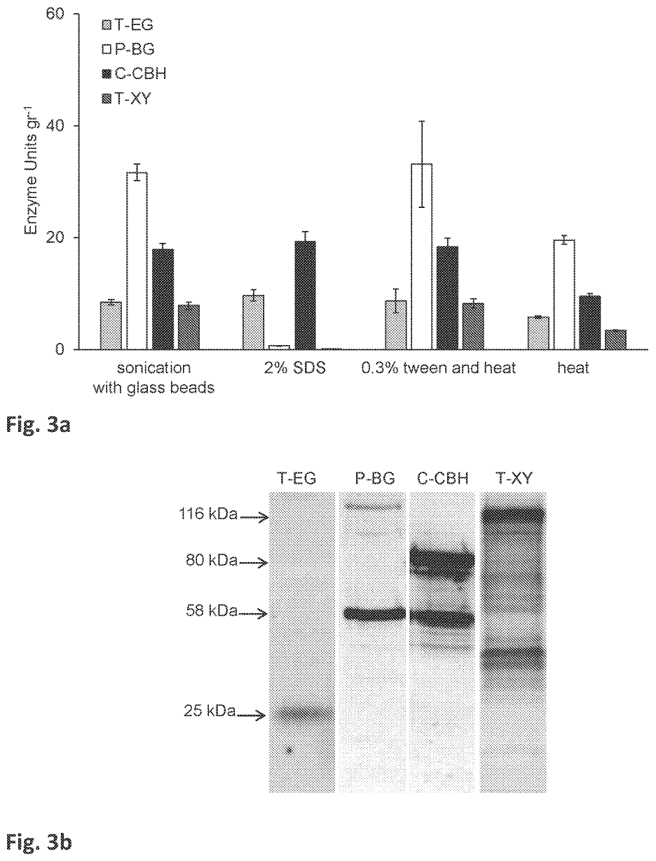

FIG. 3 shows the chloroplast expression of the HCs. a) Evaluation of the activity of the various HCs in the cell extracts of C. reinhardtii obtained by mechanical rupture (sonication+beads), by treatment with anionic detergents (2% SDS), with non-ionic detergents and heat treatment (0.3% Tween20+heat), and with heat treatment alone (heat). b) Immuno-decoration analysis carried out on cell extracts of C. reinhardtii using treatment with non-ionic detergent and heat. The enzymatic activity is expressed as Enzyme Units (μmoles min-1) per gram (DW=dry weight) of alga and was evaluated at pH 5.5 and 75° C. [T-EG: Endolgucanase of Thermotoga neapolitana, C-CBH: Cellobiohydrolase of Caldicellulosiruptor saccharolyticus, P-BG: Beta-glucosidase of Pyrococcus furiosus, T-XY: Endoxylanase of Thermotoga neapolitana].

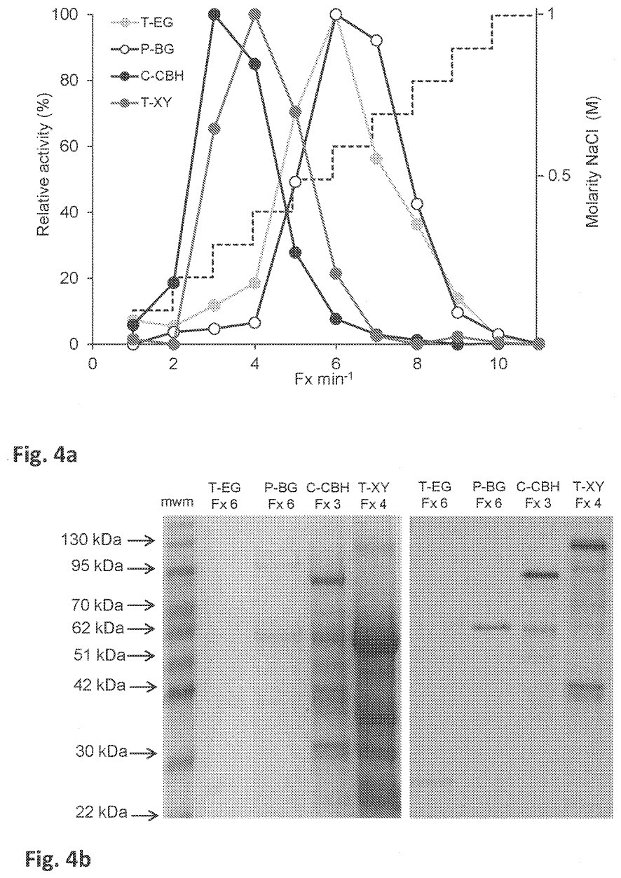

FIG. 4 shows the results of the determination of the specific activities. a) HC activity in eluted fractions (fx) from anion-exchange chromatography expressed as relative activity (%). The elution gradient to the side is also indicated. b) SDS-PAGE analysis (left) and immuno-decoration (right) carried out on the fractions that showed the greatest activity. [T-EG: Endolgucanase of Thermotoga neapolitana, C-CBH: Cellobiohydrolase of Caldicellulosiruptor saccharolyticus, P-BG: Beta-glucosidase of Pyrococcus furiosus, T-XY: Endoxylanase of Thermotoga neapolitana].

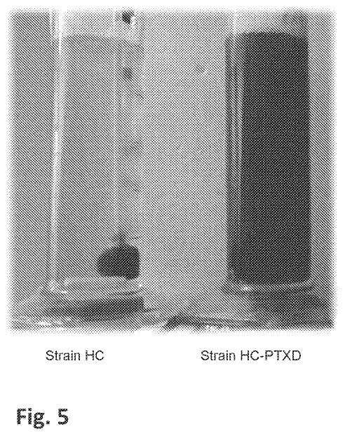

FIG. 5 shows the growth of HC-algae (HC strain) and HC-PTXD-algae (HC-PTXD strain) in mixotrophy conditions using a growth medium in which the phosphate ion was replaced with the phosphite ion.

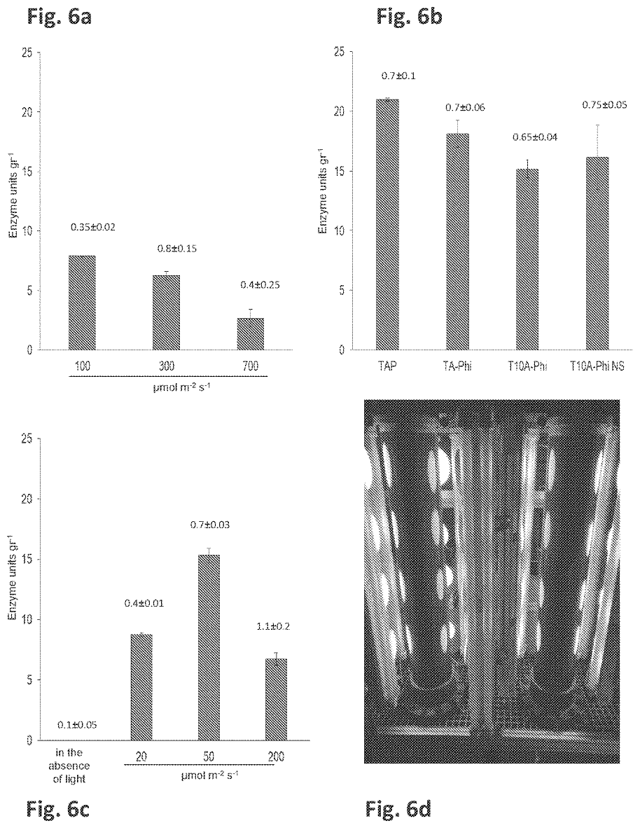

FIG. 6 shows the histograms that illustrate the results of the optimization of the expression conditions in C-CBH-PTXD algae. a) Enzymatic activity of cell extracts from algal cultures grown for seven days under photoautotrophic conditions using three different light intensities. b) Enzymatic activity of cell extracts from algal cultures grown for seven days at 50 μmol m⁻² s⁻¹ using different growth media [TAP: Tris-Acetate-Phosphate medium: TA-Phi: TAP medium in which the phosphate was replaced with 1 mM phosphite; T10A-Phi: TA-Phi with 10% of Tris; T10A-Phi NS: T10A-Phi obtained with non-sterile materials and conditions]. c) Enzymatic activity of cell extracts from algal cultures grown for seven days in T10A-Phi NS using different light intensities. The numbers above the columns indicate the biomass produced (DW=dry weigh) per litre of culture. The values were calculated as the average of two different biological replicates. The concentration of the initial inocula was 2.5×10⁵ cell mL⁻¹. d) Growth of C-CBH-PTXD in the two photobioreactors with a 60 L column each using non-sterile materials and conditions.

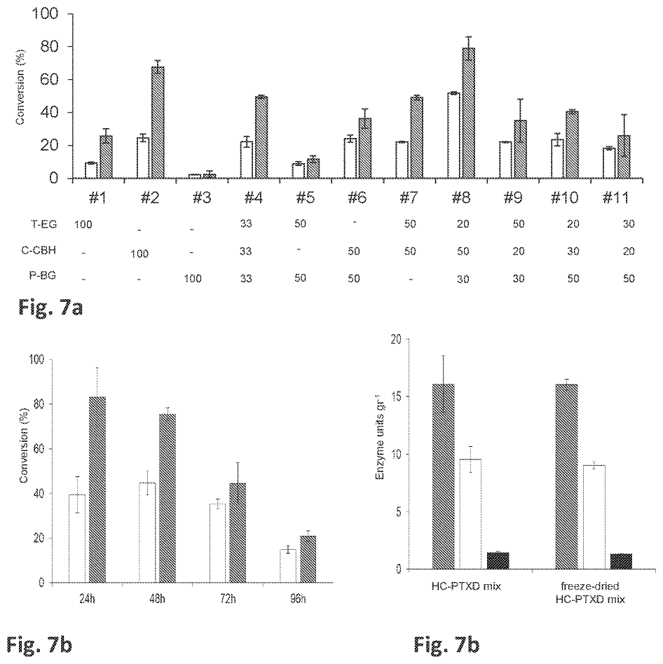

FIG. 7 shows the conversion data of PASC cellulose with protein extracts of different HC-PTXD mixtures. a) Conversion of PASC cellulose (0.6% w/v) in reducing ends (white bar) and sugars (grey bar) after 24 hours of incubation with the protein extracts of different HC-PTXD mixtures (#1-1). The numbers indicate the percentage (w/w) of the corresponding HC-PTXD strain used in each mixture. b) Conversion of PASC cellulose (0.6% w/v) in reducing ends and sugars after 24 hours of incubation with the protein extract of the HC-PTXD mixture #8. Fresh PASC cellulose (0.6%, w/v) was added to the same reaction mixture every 24 hours. Each conversion percentage refers to the last addition of PASC cellulose. c) Evaluation of the activity in the HC-PTXD mix (to which the alga T-XY, 20%, w/w was also added) against CMC (grey bar), pNPG (white bar) and xylan (black bar) before (HC-PTXD mix) and after freeze-drying and storage for 1 month at room temperature (HC-PTXD freeze-dried mix).

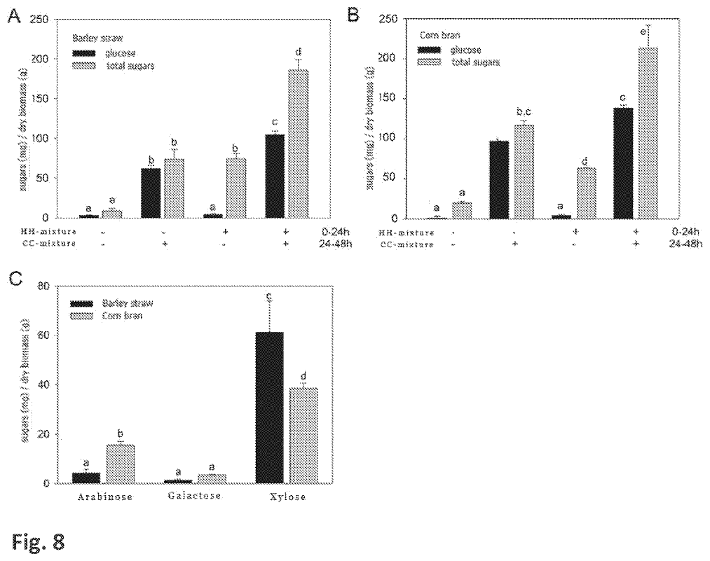

FIG. 8 shows the results of a comparison of the hydrolysis by means of cellulase (CC mixture=Celluclast+Cellobiase) and with the mixture of the invention also comprising xylanase (HH mixture=αAF+βG+ManB/5A+XynA+GghA) for the conversion of the lignocellulose substrates. Panels A and B show the release of sugars from barley straw after alkaline treatment (A) and corn bran after alkaline treatment (B) following various enzymatic treatments. The content of glucose (black bar) and total sugars (grey bar) in acid-treated filtrates were determined by GO-POD and tests with phenol-sulfuric acid, respectively. Panel C shows the analysis of the composition of monosaccharides in the filtrates treated with acids from barley straw (black bar) and from wheat straw (grey bar) after treatment with the HH mixture, as determined by HPAEC-PAD. +/−indicate treatment with active/autoclaved enzymatic mixture. The data are expressed as mean±SD, n=3. The values indicated with the same letters (a-e) are not significantly different (ANOVA test, P<0.05).

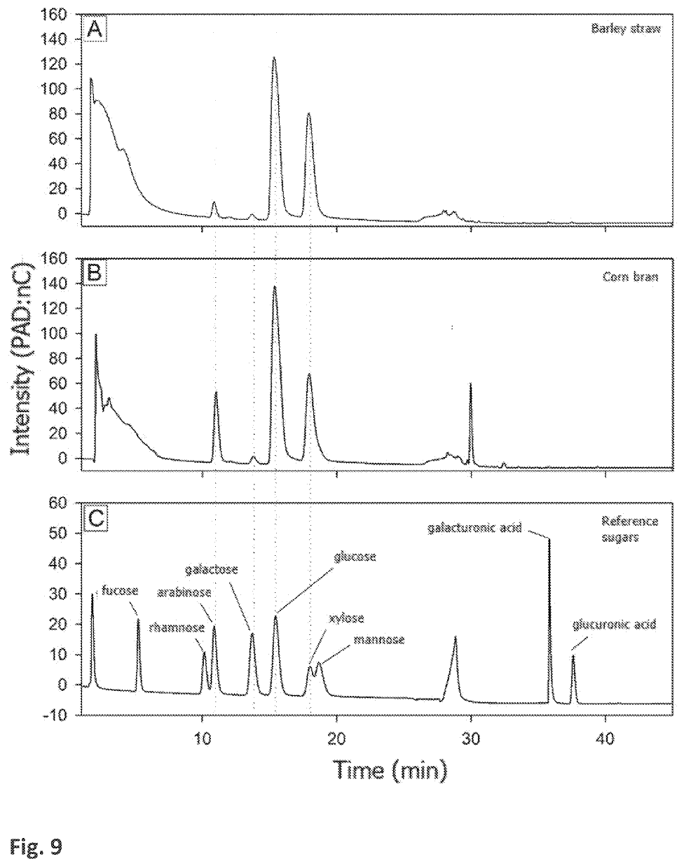

FIG. 9, shows the analysis of the composition of monosaccharides of the filtrates treated with acids, coming from barley straw and corn bran after alkaline treatment following the enzymatic reaction as determined by HPAEC-PAD. Panel (C) shows the chromatographic analysis of standard monosaccharides (HPAEC-PAD) of filtrates from barley straw (A) and corn bran (B) after treatment with HH mixtures (HH mixture=αAF+βG+ManB/5A+XynA+GghA) and CC (CC mixture=Celluclast+Cellobiase).

The following examples are now provided in order to better illustrate the invention, which are to be considered illustrative and non-limiting thereof.

### EXAMPLE 1: IN VITRO SYNTHESIS AND CLONING OF GENES ENCODING IHCS

### Materials and Methods

Chloroplast Expression of Cellulolytic Enzymes in C. reinhardtii

Endoglucanase B of Thermotoga neapolitana (T-EG) (Bok J D et al., 1998), the portion with a cellobiohydrolase activity of the cellulosome CelB of Caldicellulosiruptor saccharolyticus known as CBM3GH5 (C-CBH; Park J I et al., 2011; U.S. Pat. No. 9,624,482 B2) and beta-glucosidase of Pyrococcus furiosus (P-BG) Kengen S W M et al., 1993: Kado Y. et al., 2011) were selected as the main components of the cellulolytic machine to be expressed in the chloroplast of the microalga.

Furthermore, xylanase XynA of T. neapolitana (T-XY) (Zverlov et al., 1996) was included as a supporting enzyme for the degradation of more complex substrates (Hu J. et al. 2011) as xylan, a substrate of XynA. is one of the most abundant hemicelluloses and its presence can inhibit the cellulase activity. The four HCs were expressed individually in the chloroplast C. reinhardtii in order to compartmentalize and at the same time maximize their expression (Rochaix J. D. et al., 2014). The co-expression of the HCs in the same cell was avoided as an efficient hydrolysis reaction requires precise quantities of each enzyme and, in the case of co-expression, this cannot be managed by the operator.

In particular, the following protein sequences CelB of T. neapolitana (UniprotKB: P96492, aa 18-274) (SEQ ID NR: 1; SEQ ID NR: 2), the portion CBM3GH5 of the cellulosome CelB of C. saccharolyticus (UniprotKB: P10474, aa 380-1039) (SEQ ID NR: 3, SEQ ID NR: 4), the beta-glucosidase of P. furiosus (UniprotKB: Q51723, aa 1-472) (SEQ. ID NR: 5, SEQ ID NR: 6) and xylanase XynA of T. neapolitana (UniprotKB: Q60042, aa 30-1055) (SEQ. ID NR: 7, SEQ ID NR: 8) were converted into nucleotide sequences with codon usage optimized for chloroplast expression in C. reinhardtii using the OPTIMIZER program.

The sequences encoding the enzymes that make up the cellulolytic machine (FIG. 1) were optimized for the chloroplast expression of C. reinhardtii and fused at 3′ with the sequence encoding the HA epitope (C-terminal of the protein sequence) which allowed them to be detected by immuno-decoration analysis. The genes were subsequently cloned in an expression vector optimized for chloroplast expression (Day A. and Goldschmidt-Clermont M. 2011; Michelet L. et al., 2011) and introduced individually into C. reinhardtii by biolistic gun bombardment (Purton S., 2007). The transformants obtained were subjected to repeated selection cycles to promote the condition of homoplasmy which was then confirmed by PCR analysis on the DNA of the transformants (FIG. 2).

The possible presence of signal peptides for secretion in an extracellular environment was excluded through the use of the Signal IP 4.1 Server program. The sequence encoding the HA epitope (YPYDVPDYA) was added to the 3′ of each sequence. The sequences containing the restriction sites NcoI and SphI were fused at 5′ and 3′, respectively, of each sequence. Intragenic sequences containing the restriction sites NeoI, SphI, ClaI and SmaI were carefully mutated in order to eliminate the presence of the restriction site without altering the resulting protein sequence. The genes were then synthesized in vitro by GeneArt (Life Technologies). The synthetic genes were cloned separately downstream of the promoter that regulates the expression of the gene psaA within the vector pCLE (SEQ ID NR: 9) using the restriction sites NcoI and SphI. The expression cassette including the transgene and gene aadA that confers spectinomycin resistance was subsequently excised from the vector pCLE using the restriction sites ClaI and SmaI and transferred to the expression vector pLM20 (Michelet L. et al. 2011) (SEQ ID NR: 10). E. coli strain XL10gold (Agilent Technologies) was transformed with these constructs and used for the propagation of the recombinant DNA.

### Transformation and Selection of the Strains HC-PTXD

The four strains of HC-alga were subsequently engineered for the expression of the gene PTXD of Pseudomonas stutzeri which encodes an oxidoreductase (Costas A M G, 2001), whose expression confers to the microalga the capacity of metabolizing the phosphite ion as the sole source of phosphorus (López-Arredondo D. and Herrera-Estrella L., 2012; Loera-Quezada M M et al., 2015; US 2012/0295303 A1). The strain of C. reinhardtii used is 1a+. For the chloroplast transformation of genes expressing the HC enzymes, 1a+ was transformed using the same procedures and instrumentations described in Faè et al., 2017. The selection of homoplasmic transformants was conducted as indicated in Goldschmidt-Clermont, 1991.

For PTXD nuclear expression (SEQ ID NR. 11; SEQ ID NR: 12), HC-algae were electroporated using the same construct used in (Loera-Quezada M M et al., 2016) (SEQ ID NR: 13) and the transformants obtained (called HC-PTXID algae) were analyzed for their capacity of growing in culture media containing the phosphite ion instead of the phosphate ion. In particular, the selection of transformants expressing PTXD was carried out by monitoring the growth of transformants in a growth medium consisting of only TA (Tris Acetate) and phosphite ion at a concentration of 0.3 mM, at a temperature of 25° C. and a luminous intensity of 50 μmol m⁻² s⁻¹. The transformants capable of growing under these conditions were inoculated in growth media with an increased concentration of phosphite ion (up to 5 mM) and then selected as HC-PTXD-algae (FIG. 5).

Growth of C. reinhardtii

On a small scale, C. reinhardtii was propagated in a system of the multi-cultivator type (Photon System Instruments) at a temperature of 25° C., a luminous intensity of 50 μmol m⁻² s⁻¹ and bubbling air. The growth of the HC-PTXD.algae was carried out in modified versions of the growth media HS and TAP in which the source of phosphorus consists of different concentrations of phosphite ion from 0.3 to 5 mM. The growth medium T10A-Phi consists of 10% of the normal concentration of Tris used in the preparation of the growth medium TAP (i.e. 0.2 g L⁻¹). The growth of C. reinhardtii in a non-sterile growth medium involved the use of running tap water. For the growth of C. reinhardtii on a large scale, a 60-L column photobioreactor (produced by SCUBLA srl) was adopted in which the growth conditions that allowed the greatest productivity on a small scale (multi-cultivator system) were used.

### Optimization of the Chloroplast Expression in the HC-PTXD Microalga

With the aim of determining the growth conditions that allowed a greater accumulation of HC enzyme, the activity of C-CBH, indicated herein as an expression of a reference HC enzyme, was evaluated by cultivating the microalga under different light conditions and growth media (FIG. 6, panels a-c). The fact that the growth of the strain C-CBH-PTXD under conditions of non-sterility (e.g. using non-sterile running water) did not affect the expression levels of the C-CBH enzyme (FIG. 6, panel b) is noteworthy. Among the various light conditions tested, the lower algal biomass obtained under light conditions equal to 50 μmol m⁻² s⁻¹ (0.7 g L⁻¹) was however balanced by a higher expression level of the enzyme (15.3 U gr⁻¹) indicating that the light intensity of 50 μmol m⁻² s-1 is optimal for the expression of HC enzymes under mixotrophy conditions (10.7 U L⁻¹) (FIG. 6, panel c). The same C-CBH productivity was also obtained in 60-L column photobioreactors using a cheaper version of the TAP growth medium, consisting of 10% of the commonly used Tris concentration, as well as phosphite ion instead of phosphate ion and non-sterile running water (FIG. 6, panel d).

Protein Extraction of C. reinhardtii and Enzymatic Assays

The protein extraction was carried out using different methods and conditions from the freeze-dried cells of C. reinhardtii. After freeze-drying. the resulting powder was stored at room temperature for 1 month or, for longer periods, at −80° C. The freeze-dried algae were re-suspended in a ratio [1 mL extraction buffer:6 mg DW microalgae]. The extraction under non-denaturing conditions was carried out using a lysis buffer consisting of 10 mM citrate pH 5.5 and 0.3% Tween20. The re-suspended samples were incubated under mild stirring for 1 h at 70° C. For extraction by mechanical rupture, the cells were incubated for 30 minutes in an Ultrasonic bath (Sigma-Aldritch) bath, in the presence of glass beads with a diameter of 425-600 μm (Sigma-Aldritch). The extraction under denaturing conditions was carried out using a lysis buffer consisting of 20 mM Tris-HCl pH 7.0, 2% SDS and 10 mM EDTA. The re-suspension of the sample was carried out in a ratio [1 mL extraction buffer:6 mg DW microalgae]. After extraction, the sample was centrifuged (14.000 r×10 minutes) and the supernatant used for the analysis. The total proteins from 60 μg DW of algal biomass were analyzed by SDS-PAGE or by enzymatic assay; a monoclonal antibody AbHA (HA7 clone, Sigma-Aldritch) was used for the immuno-decoration analysis.

For the enzymatic assays, the protein extracts ( 1/10 of the total reaction volume) were incubated in a buffer consisting of 50 mM of Sodium Acetate pH 5.5 and substrate at the following concentrations: 1% CMC (to evaluate the endoglucanase activity of T-EG and cellobiohydrolase of C-CBH), 5 mM pNPG (to evaluate the beta-glucosidase activity of P-BG) and 1% xylan (to evaluate the xylanase activity of T-XY). All the substrates were purchased from Sigma-Aldritch. The pH and optimal temperature conditions for the enzymatic reactions were established on the basis of the enzymatic characterizations indicated previously in Kengen S. W. M. et al. 1993; Zverlov V. et al., 1996; Bok J. D. et al. 1998; Park J. I. et al. 2011, choosing a single pH and temperature value for conducting all of the reactions (i.e. 75° C. and pH 5.5).

The activity was expressed as Enzyme Units (μmoles reducing ends min⁻¹ or μmoles p-nitrophenol min⁻¹) per gram (g) dry weight (DW) of microalga. The determination of the micromoles of reducing ends following enzymatic hydrolysis was carried out as in (Lever M., 1972) using different quantities of glucose as a calibration curve. The determination of the μmoles of p-nitrophenol released following hydrolysis was effected using different quantities of p-nitrophenol as a calibration curve. The values of Enzyme Units were calculated as the average of two different reaction times; the same reaction carried out using autoclaved cell extracts was used as a negative control.

### Purification of HCs and Determination of the Specific Activity

For the purification of the HCs, extraction under non-denaturing conditions was carried out from 100 mg of C. reinhardtii lyophilisate using a modified buffer (10 mM Tris-HCl pH 7.5, 0.3% Tween20) in a ratio [1 mL extraction buffer:6 mg DW microalgae]. After 1 hour of incubation at 70° C., followed by centrifugation of the sample (14,000 r×10 minutes), the supernatant was charged onto a Q-sepharose chromatographic column (Amersham) pre-balanced with 20 mM of Tris-HCl pH 7.5. The elution was carried out using a NaCl step gradient. The fractions eluted from the Q-sepharose column were analyzed by activity assay. The fractions that showed the highest activity were analyzed by SDS-PAGE to determine the enzyme concentration using different quantities of BSA as calibration curve. The determination of the concentration was effected by means of the Quantity-One program (Biorad). The identity of the bands was confirmed by immuno-decoration analysis. The specific activity was used for determining the expression levels of each enzyme for each strain of HCG-algae. The specific activity of the various HCs was evaluated at 75° C. and pH 5.5.

### Evaluation of the Enzymatic Activity of HCs

The enzymatic activity of the HCs was then evaluated in cell extracts of C. reinhardtii; different extraction methods such as sonication in the presence of glass beads or treatment with anionic and non-ionic detergents were used for determining which method was the most suitable for the extraction of the enzymes. The HCs were efficiently extracted by incubating the cells at 70° C. in the presence of 0.3% (v/v) of Tween20, as shown by the levels of activity comparable to those obtained by mechanical cell rupture (FIG. 3, panel a).

The fact that the enzymes T-EG and C-CBH were resistant to SDS treatment, suggesting their possible application in reaction buffers containing anionic detergents (Li Y. et al., 2016) is noteworthy. Immuno-decoration analysis confirmed the presence of the four enzymes in the cell lysates which, as indicated by the different signal intensities, were expressed at different levels (FIG. 3, panel b). In this case, the protease inhibitors were not added to the extraction buffer in order to mimic the real field extraction and reaction conditions.

A purification procedure consisting of a thermal enrichment (Patchett M. L. et al., 1989) followed by an anion exchange chromatography (AEC) allowed the four enzymes to be purified; as they are proteins with an acid isoelectric point, they were retained by the chromatographic column under neutral pH conditions, and were eluted with NaCl concentrations ranging from 0.3 to 0.6 M.

The activity of the various enzymes was used for verifying their presence in the different fractions eluted from AEC chromatography (FIG. 4. panel a). The fractions that showed the highest activity were evaluated by SDS-PAGE analysis and immuno-decoration analysis; the latter confirmed bands with the expected molecular weight for each enzyme isolated (FIG. 4, panel b).

After determining the concentration of each enzyme in the various fractions, the specific activity (expressed as Enzyme Units per mg of Enzyme) was calculated (FIG. 4, panel c) which, in turn, allowed the level in the initial cell extracts to be estimated.

The following Table 1 shows the results of the specific activity of the various HCs towards the carboxymethyl-cellulose (CMC) 1%, paranitrophenylglucoside (pNPG) 5 mM and xylan (Xylan) 1% substrates.

TABLE 1

| U | | | | |
|---|---|---|---|---|
| mg⁻¹ | T-EG | P-BG | C-CBH | T-XY |
| CMC | 384 ± 12 | — | 23.8 ± 0.1 | — |
| pNPG | | 99.8 ± 1.4 | — | |
| Xilan | — | — | — | 52.6 ± 2.1 |

The highest yield was obtained for cellobiohydrolase C-CBH (0.8-1 mg g⁻¹ DW alga), followed by beta-glucosidase P-BG (0.3-0.4 mg g⁻¹ DW alga) and xylanase T-XY (0.2-0.3 mg g⁻¹ DW alga). The endoglucanase yield was the lowest (0.02-0.03 mg g⁻¹ DW alga) in agreement with the low signal detected by the immuno-decoration analysis on the cell extracts (FIG. 3, panel b).

It should be pointed out, however, that any contaminants in the crude cell extracts can interfere with the enzymatic activity resulting in an underestimation of the actual expression level of the enzyme.

Pretreatment of PASC Cellulose with Alga-Based Powder

The cellulose pretreated with phosphoric acid (PASC) was prepared as described in Cannella D. et al., 2016. The PASC cellulose obtained following this procedure is characterized by a sugar content (glucose) greater than 90% (w/w). 0.6 g of freeze-dried HC-PTXD mix was re-suspended in 100 mL of non-denaturing extraction buffer and incubated at 70° C. for 1 hour. At the end of the incubation, the sample was centrifuged (14,000 r×10 min) and the supernatant used for enzymatic assays. For this reason, PASC cellulose was added to the supernatant (0.3 ppure 0.6%, w/v) and the reaction was incubated at 75° C. for 24 hours. In order to test the thermal resistance of the HCs, the reaction mixture was added with fresh PASC cellulose every 24 hours. The reaction to which no new PASC was added was used as a negative control of the reaction to which PASC was added. This procedure was repeated for a total of 4 cycles of 24 hours each. Before the analysis of soluble sugars, the sample was centrifuged (4,000 r×5 min) and the supernatant used for subsequent analyses. The conversion (%) refers to the weight percentage of (reducing ends and total sugars) released by the PASC cellulose.

### Determination of Carbohydrates in PASC Cellulose

The determination of the μmoles of reducing ends released following enzymatic hydrolysis was effected in accordance with (Lever M., 1972) using different quantities of glucose as a calibration curve. The total sugars were determined by the colorimetric assay of phenol-sulfuric acid (Dubois M. et al., 1956). The total sugars contained in the PASC cellulose were determined after acid hydrolysis of the substrate carried out following the procedure described by the Laboratory Analytical Procedure of the National Renewable Energy Laboratory: the sample was first dissolved in 72% sulfuric acid (v/v) at a temperature of 30° C. for 1 hour and was then diluted to a final concentration of sulfuric acid of 4% (v/v) and incubated at a temperature of 120° C. for 1 hour. The supernatant was then tested to determine the quantity of solubilized sugars. The sugars were then determined by the colorimetric test of phenol-sulfuric acid. The values reported are an average of three independent replicates (Dubois M. et al., 1956).

### Optimization of the HC-PTXD Mixture

In order to optimize the algae-based product for the degradation of lignocellulosic material, different mixtures of the various strains expressing the HC enzymes (i.e. HC-PTXD-algae) were tested and also their stability following long-term storage. It should be pointed out that the enzymes selected are all characterized by an optimal pH ranging from 5 to 6 which allowed their simultaneous use without significant activity losses (Kengen S W M et al. 1993; Zverlov V. et al., 1996; Bok J D et al. 1998: Park J I et al. 2011). The degradative capacity of the extracts obtained from 11 different mixtures of the four HC-PTXD-algae was tested using, as substrate. cellulose pretreated with phosphoric acid, whose acronym is PASC. After 1 day of incubation at 75° C., the highest conversion of the PASC substrate into soluble sugars (expressed as total sugars and reducing agents) was obtained from mixture #8 (FIG. 7, panel a), whose composition [T-EG:C-CBH:P-BG] is 20:50:30 (w/w/w). The formulation HC-PTXD #8, hereinafter referred to as HC-PTXD mix, is characterized by a 0.1% cellulolytic enzyme content (w/w: 5% T-EG, 66% C-CBH, 29% P-BG corresponding to 0.01 mg of T-EG, 0.5 mg of C-CBH and 0.15 mg of P-BG for g DW alga).

In order to also determine the thermo-resistance of the HC enzymes, fresh PASC was added to the cell extracts every 24 hours for a total period of four days. The enzymatic activity remained unchanged until the third addition of PASC, indicating that the enzymes remained stable until the third day of reaction (FIG. 7, panel b).

In these experiments. HC-PTXD mix algae powders were used, obtained by freeze-drying, and which were then stored at room temperature for a month before being used. Neither the freeze-drying procedure nor the storage conditions altered the functionality of the enzymes, indicating that the C. reinhardtii chloroplast is an effective compartment for the preservation of cellulolytic enzymes with a heat-stable activity (FIG. 7, panel c).

A further analysis of the substrate specificity of the HC-PTXD mix showed that the mixture is also active against microcrystalline cellulose, even if at a lower level (i.e. Avicell PH101).

The following Table 2 indicates the specific activity of the HC-PTXD mix towards the different cellulose substrates CMC 1% (w/v), PASC cellulose 0.6% (w/v) and Avicell 2.5% (w/v). The enzymatic activity is expressed as Enzyme Units (μmoles min-1) per gram (dw) of HC-PTXD mix and is calculated at pH 5.5 and 75° C.

TABLE 2

| Enzyme Units gr⁻¹ | CMC | PASC | AVICELL |
|---|---|---|---|
| HC-PTXD mix | 16.1 ± 3.25 | 3.1 ± 0.21 | 0.6 ± 0.04 |

### EXAMPLE 2: THE ACTIVITY OF XYNA XYLANASE ENHANCES THE CELLULASE ACTIVITY ON LIGNOCELLULOSIC SUBSTRATES

### Materials and Methods

The barley straw (Hordeum vulgare) was supplied by Prof. Felice Cervone (Department of Biology and Biotechnology, Università La Sapienza, Rome). The corn bran (Zea mays) was supplied by Prof. David Bolzonella (Department of Biotechnology, University of Verona). In both cases, the lignocellulosic material was pre-treated with a mild alkaline solution. The material was homogenized in liquid nitrogen and mixed with 0.1 g of NaOH per g of substrate in an appropriate volume of water to give a 4% NaOH solution. The samples were incubated at 75° C. for 2 hours and the insoluble solid fraction was washed several times with ultrapure water before freeze-drying and storage at room temperature. For the enzymatic assays. this insoluble solid fraction (1.5% w/v) was incubated in a 50 mM citrate-phosphate buffer (pH 6) with a 0.5% (v/v) Celluclast/Cellobiase mixture (CC mixture) or with a mixture of 0.1 mg mL⁻¹ of hemicellulase supplemented with 0.02% NaN₃ (HH mixture). The CC mixture comprises 0.4% (v/v) of cellulase from Trichoderma reseei (Celluclast) and 0.1% (v/v) of cellobiase from Aspergillus niger (Cellobiase) of Sigma-Aldrich. The HH mixture comprises equimolar quantities of each thermophilic hemicellulase enzyme from T. neapolitana and in particular

αAF: alpha-arabinofuranosidase, degrades arabinose from polysaccharides such as galactan and xylan
βG: beta-endogalactanase, degrades galactan (consisting of galactose)
ManB/5A: beta-endomannanase, degrades mannan (consisting of mannose)
XynA: beta-endoxylanase, degrades xylan (consisting of xylose)
GghA: beta-glucan glucohydrolase, degrades some disaccharides

A two-step enzyme degradation was also carried out in which the sample was incubated first at 75° C. for 24 hours with the HH mixture and then at 37° C. for another 24 hours with the CC mixture.

Inactivated enzymatic mixtures were added at the same time as negative controls. The yield of soluble sugars was evaluated at the end of the reaction using the phenol-sulfuric acid assay. (Dubois M. et al., 1956). The hydrolysis of the substrate was indicated as a quantity of sugars (g) released in proportion to the weight of the insoluble lignocellulosic material treated with alkalis (g) and expressed in percentage terms. The glucose in the acid-neutralized filtrates was quantified using the glucose oxidase/peroxidase (GOPOD) assay kit of Megazyme.

The composition of the monosaccharides of the lignocellulosic materials treated with alkalis and the reaction filtrates was determined in samples neutralized with acids by means of high-performance anion exchange chromatography coupled with pulsed amperometric detection (HPAEC-PAD).

### Results

FIG. 8 shows the comparison data of the hydrolysis of lignocellulose substrates by means of a mixture of cellulases (CC mixture=Celluclast+Cellobiase) and with a mixture of hemicellulases also comprising xylanase XynA (HH mixture=αAF+βG+ManB/5A+XynA+GghA).

This analysis shows that pretreatment with hemi-cellulases is able to enhance the action of cellulases; this can be deduced in FIG. 8, panels A-B comparing the release of glucose (black bar) by cellulases when the material has been pretreated with hemicellulases (+, +) compared to that released when the material was treated with cellulases alone (−, +).

In FIG. 8, panel C, the sugars released by the pretreatment with hemicellulase are analyzed and there is a marked prevalence of xylose (the final product of the degradation of xylan, the substrate of XynA) in both lignocellulosic substrates (barley straw and corn bran).

FIG. 9, on the other hand, shows the results of the qualitative and quantitative analysis by means of HPAEC-PAD chromatography of the hydrolyzates of lignocellulosic materials (barley straw and corn bran) following treatment with hemicellulase and cellulase. The analysis shows that the increased release of glucose (the degradation product of cellulases) is always associated with a marked release of xylose (the constituent of xylan, substrate of XynA) in both lignocellulosic substrates treated with enzymatic mixtures.

These results on the whole show how the increased release of glucose by cellulases can be attributed to the synergistic action of the addition of xylanase.

### BIBLIOGRAPHY

Anitori R P (2012). Extremophiles: microbiology and biotechnology. Caister Academic Press.
Badici M, Asim N, Jahim J M, Sopian K (2014). APCBEE Procedia 9:170-174.
Benedetti M, Verrascina I, Pontiggia D, et al (2018). Plant J.
Blifemez-Klassen O, Klassen V, Doebbe A, et al (2012). Nat. Commun. 3:1214.
Bok J D, Yernool D A, Eveleigh D E (1998). Appl Environ Microbiol 64:4774-4781.
Brasil B dos SAF, de Siqueira F G, Salum T F C, et al. (2017). Algal Res 25:76-89.
Cannella D, Möllers K B, Frigaard N U, et al (2016). Nat Commun 7.
Costas A M G, White A K, Metcalf W W (2001). J Biol Chem 276:17429-17436.
Day A. Goldschmidt-Clermont M (2011). Plant Biotechnol. J. 9:540-553.
Dubois M, Gilles K A, Hamilton J K, et al. (1956) Anal Chem 28:350-356.
Faè M. et al. (2017) ppl Microbiol Biotechnol 101:4085-4092.
Goldschmidt-Clermont M. (1991). Nucleic Acids Res 19:4083-4089.
Harmsen P F H, Huijgen W J J, Bermnudez López L M, Bakker R R C (2010) Literature Review of Physical and Chemical Pretreatment Processes for Lignocellulosic Biomass. Ed: Wageningen U R. Food & Biobased Research. 1-54.
Hu J, Arantes V, Saddler iN (2011) Biotechnol Biofuels 4:36.
Jönsson Li, Martin C. (2016). Bioresour. Technol. 199:103-112.
Juge N. (2006). Trends Plant Sci 11:359-367.
Kado Y, Inoue T, Ishikawa K (2011). Acta Crystallogr Sect F Struct Biol Cryst Commun 67:1473-9.
Kalunke R M, Tundo S, Benedetti M. et al (2015). Front Plant Sci.
Kengen S W M, Luesink E J, Stains A J M, Zenhder A J B (1993). Eur J Biochem 213:305 312.
Kumar A K, Sharma S (2017). Bioresour Bioprocess 4:7.
Lever M (1972). Anal Biochem 47:273-279.
Li Y, Sun Z, Ge X, Zhang J (2016). Biotechnol Biofuels 9:20.
Loera-Quezada M M, Leyva-Gonzalez M A, López-Arredondo D, Herrera-Estrella L (2015). Plant Sci 231:124-130.
Loera-Quezada M M, Leyva-González M A, Velázquez-Juárez G, et al (2016). Plant Biotechnol J 14:2066-2076.
López-Arredondo D L, Herrera-Estrella L (2012) Nat Biotechnol 30:889-893.
Mayfield S P, Manuell A L, Chen S, et al (2007). Curr Opin Biotechnol 18:126-133.
Michelet L, Lefebvre-Legendre L, Burr S E, et al (2011) Plant Biotechnol J 9:565-574.
Ooshima H, Sakata M, Harano Y (1986). Biotechnol Bioeng 28:1727-1734.
Park J I, Kent M S, Datta S, et al (2011). Bioresour Technol 102:5988-5994.
Patchett M L, Neal T L, Schofield L R, et al (1989). Enzyme Microb Technol 11:113-115.
Peng X. Qiao W, Mi S. et al (2015). Biotechnol Biofuels 8:131
Purton S (2007). Adv. Exp. Med. Biol. 616:34-45.
Rasala B A, Lee P A, Shen Z. et al (2012). PLoS One 7:e43349.
Rochaix J D, Surzycki R, Ramundo S (2014). Methods Mol Biol 1132:413-424.
Rodolfi L. Chini Zittelli G. Bassi N. et al (2009) Biotechnol Bioeng 102:100-112.
Saini J K, Saini R, Tewari L (2015). Biotech 5:337-353.
Sánchez C (2009). Biotechnol Adv 27:185-194.
Sanderson K (2011). Nature 474:S12-4.
Sarmiento F, Peralta R, Blarney J M (2015). Front Bioeng Biotechnol 3:148.
Schroda M. (2006). Curr Genet 49:69-84.
Souza T V., Araujo J N, Da Silva V M, et al (2016). Biotechnol Reports 9:1-8.
York W S, Qin Q. Rose J K. (2004). Proteins Proteomics 1696:223-233.
Zverlov V, Piotukh K, Dakhova O, et al. (1996). Appl Microbiol Biotechnol 45:245-247.

1. A combination of transgenic microalgae wherein each transgenic microalga expresses a phospite dehydrogenase D of a bacterial origin and a heat-stable plant cell wall degradative enzyme selected from the group consisting of endoglucanase B of Thermotoga neapolitana (SEQ ID NR: 1), the portion with a cellobiohydrolase activity of the cellulosome CelB of Caldicellulosiruptor saccharolyticus (SEQ ID NR: 3) and beta-glucosidase of Pyrococcus furiosus (SEQ ID NR: 5), wherein said endoglucanase B of Thermotoga neapolitana is encoded by the nucleotide sequence with codon usage optimized for the chloroplast expression SEQ ID NR: 2, said portion of the cellulosome CelB of Caldicellulosiruptor saccharolyticus is encoded by the nucleotide sequence with codon usage optimized for the chloroplast expression SEQ ID NR: 4, and said beta-glucosidase of Pyrococcus furiosus is encoded by the nucleotide sequence with codon usage optimized for the chloroplast expression SEQ ID NR: 6.

2. The combination of transgenic microalgae according to claim 1, further comprising a transgenic microalga expressing a phosphite dehydrogenase D of a bacterial origin and xylanase XynA of Thermotoga neapolitana (SEQ ID NR: 7).

3. The combination of transgenic microalgae according to claim 2, wherein said xylanase XynA of Thermotoga neapolitana is encoded by the nucleotide sequence with codon usage optimized for the chloroplast expression SEQ ID NR: 8.

4. The combination of transgenic microalgae according to claim 1, further comprising a transgenic microalga expressing a phosphite dehydrogenase D of a bacterial origin and a ligninase selected from laccase of Thermus thermophilus (SEQ ID NR: 14) and polyphenol oxidase of Thermus thermophilus (SEQ ID NR: 16).

5. The combination of transgenic microalgae according to claim 1, belonging to the species Chlamydomonas reinhardtii.

6. The combination of transgenic microalgae according to claim 1, wherein said phosphite dehydrogenase D comes from Pseudomonas stutzeri (SEQ ID NR: 11) and is encoded by the nucleotide sequence SEQ ID NR: 12.

7. Use of a combination of transgenic microalgae according to claim 1, for the production of a mixture of heat-stable plant cell wall degradative enzymes in a culture medium comprising the phosphate ion as sole phosphorous source.

8. A process for the production of a mixture of heat-stable plant cell wall degradative enzymes, comprising the following steps:
a) cultivating a combination of transgenic microalgae according to claim 1 in a culture medium comprising the phosphate ion as sole phosphorous source, in a photobioreactor;
b) freeze-drying of the microalgae;
extracting the enzymes alternatively by sonication, short heat treatment at 80° C., non-denaturing conditions or denaturing conditions.

9. A mixture of heat-stable plant cell wall degradative enzymes obtained according to the process of claim 8, said mixture comprising endoglucanase B of Thermotoga neapolitana (SEQ ID NR: 1), the portion with a cellobiohydrolase activity of the cellulosome CelB of Caldicellulosiruptor saccharolyticus (SEQ ID NR: 3), beta-glucosidase of Pyrococcus furiosus (SEQ ID NR: 5), in a ratio [g strain endoglucanase B:g strain cellobiohydrolase portion of the cellulosome CelB:g strain beta-glucosidase] of 20:50:30.

10. A mixture of heat-stable plant cell wall degradative enzymes obtained according to the process of claim 8, said mixture comprising endoglucanase B of Thermotoga neapolitana (SEQ ID NR: 1), the portion with a cellobiohydrolase activity of the cellulosome CelB of Caldicellulosiruptor saccharolyticus (SEQ ID NR: 3), beta-glucosidase of Pyrococcus furiosus (SEQ ID NR: 5), and xylanase XynA of Thermotoga neapolitana (SEQ ID NR: 7), in a ratio [g strain endoglucanase B:g strain cellobiohydrolase portion of the cellulosome CelB:gstrain beta-glucosidase:g strain xylanase] of 20:40:20:20.

11. The mixture of heat-stable plant cell wall degradative enzymes according to claim 9, further comprising in addition or alternatively a ligninase selected from laccase of Thermus thermophilus (SEQ ID NR: 14) and polyphenol oxidase of Thermus thermophilus (SEQ ID NR: 16).

12. The mixture of heat-stable plant cell wall degradative enzymes according to claim 9, in the form of freeze-dried powder.

13. Use of the mixture of heat-stable plant cell wall degradative enzymes according to claim 9, for the biodegradation of cellulose-based or lignocellulose-based substrates.

14. Use of xylanase XynA of Thermotoga neapolitana (SEQ ID NR: 7) in a mixture with heat-stable plant cell wall degradative enzymes comprising endoglucanase B of Thermotoga neapolitana (SEQ ID NR: 1), the portion with a cellobiohydrolase activity of the cellulosome CelB of Caldicellulosiruptor saccharolyticus (SEQ ID NR: 3) and beta-glucosidase of Pyrococcus furiosus (SEQ ID NR: 5) as preventive treatment for the biodegradation of lignocellulose-based substrates.
